SEQUENCE LISTING (4 sequences)

SEQ ID NO: 1 - DNA, 1572 bp, Halomonas elongata
  source 1..1572: mol_type = genomic DNA
atgaagcaaaagacggcagtcggccctctggttggcgggatgttgcttgcctcggtcgcggcgccggccagtgccgccgaggtcaagaacgtgatcctgatgatcggggacggcatggggccccagcaggtggggctattggaaacctatgccaaccaggctcccgattccatctacgatggcgagccgacggcgtttcaccaattggccaaggaaggcgtcgtcggtttttccctgacgcatcccgaggatgccgtcgtcgtggattccgcctgttccgccacgcagttggcgagtggcatctactccggtagcgaggtgatcggtatcgatgccgagggcaatcccgtcgagacggtactcgagcttgcccaggcccggggcaaggcgaccggtctggtttcggacactcgcctgacccatgccacgcccgcagccttcgccgcccaccagccccatcgttcgctggagaacgagatcgccgtggacatgctggaagtagggcccgacgtcatgctctcgggcggactgcgccattgggtgccgcaattggcgagcgaggatgccgaggtcacgagcctgatggatggcgcttatgagccggcgtccaagcgtcaggacgatcgaaacctgctcgcggaagccgtcgaaaagggctatggcctggcgttcagccgcgagcaactcgaggccgatcagagcgacaagctactgggcctgtttgccaactccggcatggccgatggtatcgagtatcgcaataccagggacgacgccgaccgcctcgagccgaccctgcacgagatgacgcaggccgcgctgaacaggctcgagcaggacgaggacggtttcttcctgatggtcgaaggcggtcagatcgactgggcggggcactcgaacgatgccggcaccatgctcaacgagatggtcaagttcgaggaggcggttcaaggtgtctacgactgggccaaaggacgcgaggacaccgtgatcctggttaccgcggatcatgaaacgggcgcgttcgggctcagttattcatcggccgacctgccggagccgcagtcgaagtcgggccctgccttcgcggaacgggactatgcgcccaacttcaacttcggcgacttcgcgcttctcgattccctctatcagcagaaggccagcttttcgaccctgctgagtgagttcggggcactcgaagaagaacagagaacgccggctcgtctgatggaaatggtcaatgccaacagcgatttccagatcgacgaagagcaggcggaagcggtcctggccgataagccgaacccctaccacgtggagggtcacagctatctcgaagccgaggaggtcccggccatccaggactttgatgcgttctacccctataacgatcggggcaatgtactggggcgtgttctgggtactgcacagaatgtcgtctggggcaccggtacccatacccacactccggtgaatgtcttcgcctgggggccggccgagaccattctgccggtatcctcgatccagcatcattccgaggtcggtcagtacctcaagtcgctggtcgagtga

SEQ ID NO: 2 - DNA, 852 bp, Halomonas elongata
  source 1..852: mol_type = genomic DNA
atgagccagagccccgtgacccagagacagtactacgccccccacggcggccatccgggacagaaggtcatgaccaccgaccgggccatgttcaccgaggctttcgcggtgattcccaagggggtctatcgcgatatcgtgaccagcaagttgccccattgggagaacacccggctgtggatactgtcccggccgctctccggttttgcggagaccttctcccagtacatcatggaggtctcgccaggcggcggcagcgaacggcccgagaccgatccgggagccgagggtgtgctgttcgtggtggagggcgagctggtgctgaccctgggcggcgagcggcatcgcatggtgcccggcggttatgccttcattccgccgggcagcgactggcaattgcgcaacgaagccgatgagccggtacgttttcactggattcgcaaggcctacgaattcgtcgacggcatcgacgttcccgaggccttcgtggtcaacgaaaatgacatcgagccgacggtgatgccggactgcaacgaggtctgggccaccacgcgcttcgtcgatccggacgacatgcgccacgatatgcacgtgaccatcgtcaccttccagccgggcggggtgattcccttcgacgagacccatgtcatggagcatggtctttacgtgctcgagggcaaggcggtctatcacctcaaccagcagtgggtggaggtcgaggccggggactacatgtggctgcgcgccttctgcccccaggcctgctatgccggtggcccagggcccttccgctatctgctttacaaggacgtcaaccggcacatggcgctgccttacagcaagcgtcgctga

SEQ ID NO: 3 - DNA, 711 bp, Halomonas elongata
  source 1..711: mol_type = genomic DNA
atggccggctccctgcctctctacctcaagatacaacggcacctgctggagaagattcatggcggcgactggccaccgcaatatcggattccccccgaggaacgtctggccgaggagttcggcgtcagccgcatgaccgccaacaaggccatccgcgacctggtacagaaagggtatctgacacgccagccgggggtcggcaccttcgtcaccgaccgcaaggccgagtcctcgttgatcgaggtgcacaatatcgccgaggaagtccgcagccgtgaccacgcctataccagcgaagtcctgcgtgccgaagcgctggaagccgacgacgagatctcggtgtatctgggggtccgcaacggtgcccgggtctttcacaccctgatcgtgcatcgcgaagacggcgtgcccatccagcttgaggatcgctacgtcaaccccagccgggtaccccactacctcgagacggacttcacgcagcacacgcccaacgaggttctggtggcagcctgcccgatcaacgacgtggagcacgtggtcgaggccgtgctggtcgacgccgacaccgcgcaacgcctcgacatcgccccccacgaaccctgcctgcgaatgatccggcgcacctggtcgggagatcacctgatcagctacgcccgcctgatccaccccggcgaccgctacaagctacgctcctcgttgcataccaactga

SEQ ID NO: 4 - DNA, 1185 bp, Halomonas elongata
  source 1..1185: mol_type = genomic DNA
atgctgacaagcacccaggaacgcatcatcgaagcgactgctcccgtcgttgccgaacatctcaccaccatcacccagcatttctatcccttgatgttcgagcgctatcccgaagtcgcgccgctcttcaaccagacccaccaggccactggcggccaaccgcgcgccctggccagctcggtgctggcatatgtccagctacgcaagaacccggcccaggctcgccatgccctggaaaccgtggtcaacaagcacgtatcgctggatatccaacctgaccagtatccgatcgtcggtgaatgcctgatggccgccatcggcgacgtcctgggcgaagccgtcaccgaagaggtcgccgatgcctggggcgcactgtatcaggagctggccgatctgctgatcgacctagagagccaacgctatcgcgaattcgccgaggcccccggcggctggcgcggagccaggtccttccgtatcgccgacatccgtcaggaaagcgccgtcattcgctccttcgttctggaacccgaagatggccagcccgttgccgacttccgccccggccagttcatcggtgttcgcctcactatcgacgacaagccggtctatcggcactacagcctgtcggccttccccaatggccgcagctaccggatctccatcaagcgcgaagccgacggcgtcgtcagccgccatttccatgacgtgctgaacatcggcgaccgcatcgagctgctgccacccgccggcgatctgaccctgccggaagaaggacacgacccgctcatgctgctcagcggcggcgtcggccagacccccatgctgtcgctgctgcgccaggccctggaacaggggcggcaggtcgtctacctgcatgcggcgctggacgaggaccatcatgcattcggcaacgacgtgcaggccctggctcgcgactatcccgatcaactgcgggtggtcaccgtctacgaacaagctgacgatgccagccatgtcgaccatatcgggcgtgtggaccgtgagctgatcgcccgctacctccctgccggcgcgcctcactgctattacgtcgggccgcagggcttcatggtcgccgtcgaccaaatcctgcaatccctgggggttcccgcagaacgccgccatttcgagcacttcgggccctcgcgcgcgctcgaggccgcctga

(19) United States
(12) Patent Application Publication
(10) Pub. No.: US 2025/0333573 A1
(43) Pub. Date: Oct. 30, 2025

(54) METHOD FOR TEXTILE AND POLYESTER DECONSTRUCTION AND UPCYCLING TO BIOPLASTIC IN A HALOPHILE

(71) Applicant: Battelle Memorial Institute, Columbus, OH (US)

(72) Inventors: Jacob Lilly, Columbus, OH (US); William R. Henson, Columbus, OH (US); Albert J. Darling, Columbus, OH (US)

(21) Appl. No.: 19/038,598

(22) Filed: Jan. 27, 2025

Related U.S. Application Data
(60) Provisional application No. 63/625,661, filed on Jan. 26, 2024.
(60) Provisional application No. 63/563,295, filed on Mar. 8, 2024.

(51) Int. Cl. C08J 11/14; C12N 9/02; C12N 15/74

(52) U.S. Cl. CPC C08J 11/14; C12N 9/001; C12N 9/0071; C12N 15/74; C12Y 103/01053; C12Y 114/12015; C08J 2301/02; C08J 2367/02

(57) ABSTRACT

Methods for deconstructing polyester-cotton blended textile waste and biodegradable polyester packaging waste using microbial cells for the fermentative production of polyhydroxybutyrate (PHB), novel engineered Halomonas strains, and use of these strains in PHB production. An engineered microbial cell and compositions comprising PHB are also described.

### CROSS REFERENCE TO RELATED APPLICATIONS

This application claims priority to U.S. Provisional Patent Applications Nos. 63/625,661 filed 26 Jan. 2024 and 63/563,295 filed 8 Mar. 2024.

### REFERENCE TO A SEQUENCE LISTING

This application contains a sequence listing in computer readable form, which is incorporated herein by reference.

### FIELD OF THE INVENTION

This invention is in the field of textile deconstruction and polyester degradation and upcycling to a bioplastic value added product using an engineered halophile.

### INTRODUCTION

The versatility of plastic materials has made them an inseparable part of varied products such as its use in the textile industry and as part of single-use packaging g materials. Many clothing textiles are composed of interwoven synthetic polyester (polyethylene terephthalate or PET) and organic fiber blends that need to be mechanically reduced; however, mechanical reduction of blended fabrics require highly sorted and pre-selected waste to be effective, and are often plagued by high processing costs, low product value, and complications with dyed fabrics which prevent their reprocessing in new clothing manufacturing. As a result, there are limited end products of value that can be generated economically from blended fabric, and the overwhelming majority end up in landfills (87%), making fast fashion the second most polluting industry globally.

Methods of recycling polyester-cotton blend textile waste have been proposed which involve chemically separating the cotton from the polyester. Methods in US 2023/0416491 A1, US 2023/0416492 A1 and U.S. Pat. No. 10,501,599 B2 employ a depolymerization reaction to dissolve the PET fibers and thereby separate the PET from the cotton fibers. The known depolymerization processes involve several steps, require high temperatures and not economically feasible when scaled-up. For example, many such processes focus on producing PET monomers (US 2023/0416492 A1, US 2024/0002628 A1), or producing TPA, a precursor of PET (U.S. Pat. No. 10,501,599 B2, US 2023/0416491 A1). The separated cotton is degraded to a cellulosic level, which requires reconstitution. Such processes require cost-prohibitive additional steps in order for the cotton to be reusable; otherwise the cotton could be destroyed. In some cases the cotton is degraded to a cellulosic material that has to be reconstituted in order to make a viscose fiber (U.S. Pat. No. 10,501,599 B2, US). Moreover, the PET monomers have limited usage in new products and are primarily used for plastic water bottles. Some of these methods produce PET that is used in lower grade fibers that are used in some textiles. As a result, these processes do not make the most efficient use of the separated cotton and PET. Upcycling of plastic through tandem chemical conversion and biofunneling has been described (Sullivan et al., Science 378, 207-211 (2022). However, an integrated process to deconstruct and upcycle blended textile waste to a high value product that is efficient and economical is desired. Such processes are necessary to significantly reduce environmental impact and improve the current state-of-the-art.

Single use plastics (e.g. in packaging) account for 50% of the plastics produced every year. To reduce environmental impact, the use of compostable flexible packaging structures are an environmentally friendly alternative to polyolefin-based packaging derived from petroleum-based polymers such as polyethylene (PE), polypropylene (PP), or polyethylene terephthalate (PET). While non-renewable polymers may provide good strength, barrier, and/or printability characteristics during their functional lifetime, at end-of-life, such polymers do not readily decompose after disposal-either in landfills or the natural environment. Thus film structures and bags made from such polymers persist for decades after disposal. Therefore recent initiatives have emerged, proposing greener alternatives, notably in packaging applications.

Aliphatic polyesters such as poly lactic acid (PLA) or polyhydroxybutyrate (PHB) are commercially available and sustainable alternatives to non-biodegradable plastics and can be readily accessible via controlled chain-growth ring-opening polymerization of cyclic esters or lactones. Despite its green credentials, one challenge however is to demonstrate recyclability by the generation of high value monomers, towards the ultimate goal of maximizing the circular plastics economy. Mechanical recycling for reprocessing of plastic packaging requires the bag to bag sorting of multi-layer packaging. However the process is limited by eventual material downcycling due to harsh remelting conditions for thermomechanical degradation (J. Payne, P. Mckeown, M. D. Jones, Polym. Degrad. Stab. 2019, 165, 170-181.) Chemical transformation by hydrolysis, transesterification, hydrosilylation, etc to recapture monomers or to directly convert to feedstock suffers from a requirement to mechanically separate the polymer mixture before chemical recycling. Additionally, chemical recycling of aliphatic polyesters by pyrolysis is energy intensive and not suitable due to the presence of oxygen. Several species of bacteria and fungi have been identified with enzymatic depolymerization properties to biodegrade PLA and PHB (https://ami journals.onlinelibrary.wiley.com/doi/10.1111/lam.13287). Under industrial composting conditions, PLA biodegrades into CO₂ and H₂O and the complete biodegradation has been reported within 30 days (https://onlinelibrary.wiley.com/doi/epdf/10.1002/mabi.200600168). Conversely, in domestic composters, PLA biodegradation can take a year or up to 12 weeks depending on composting conditions. (https://link.springer.com/article/10.1023/A: 1022849813748) (https://www.sciencedirect.com/science/article/abs/pii/S0926669010003511?via % 3Dih ub). Waste streams stemming from food waste, oil and sugar industries have been successfully used as feedstock in bioreactors to generate PHB, using different types of microorganisms (https://www.sciencedirect.com/science/article/pii/B9780128200841000016). Amongst the variety of substrates used thus far to generate PHB, glucose remains the most predominant carbon source (usually obtained by the hydrolysis of complex molecules). However, a process and method for the generation of usable carbon sources from biodegradable plastics and its upcycling is required.

Upcycling faces challenges when contaminants including food, are mixed with plastic waste. Consequently, food contamination of plastics reduces recycling/upcycling efforts, contaminates waste streams and can result in valuable recyclable material ending up in landfills.

Against this background, it is clear that biodegradable plastics can become potential contributors to plastic pollution if irresponsibly left to the natural environment to degrade. Biodegradable polymers, likely the dominant player in the future plastics economy, and polyesters in the textile industry, which are primary contributors to environmental pollution need to find sustainable methods and processes and overcome the obstacles of food contaminants to convert waste to high value products.

### SUMMARY OF THE INVENTION

In a first aspect, the invention provides a method for deconstructing polyester cotton blended textile comprising: alkaline hydrothermal treatment, conducted at a pH of more than pH 7 and at a temperature of at least 180° C., of polyester cotton blended textile to generate TPA and EG from the polyester, depolymerizing cotton, forming breakdown products; and contacting the breakdown products with bacteria of the genus Halomonas in a fermentation bioreactor.

In another aspect, the invention comprises a method for deconstructing biodegradable polymer, comprising: alkaline hydrothermal treatment of polymer to generate breakdown products comprising LA, 3HB and CA, and contacting the breakdown products with bacteria of the genus Halomonas in a fermentation bioreactor.

In a further aspect, the invention provides a method for deconstructing a mixture comprising polyester, biodegradable polymer and food comprising: alkaline hydrothermal treatment, conducted at a pH of more than pH 7 and at a temperature of at least 180° C., forming breakdown products; and contacting the breakdown products with engineered bacteria of the genus Halomonas in a fermentation bioreactor.

In another aspect, the invention provides an engineered microbial cell that utilizes TPA to produce PHB. In a further aspect, the invention provides a microbial cell that utilizes hydrothermal reaction products of aliphatic polyester to produce PHB

The invention can be further characterized by one or any combination of the following: wherein the alkaline hydrothermal treatment comprises removing dyes from blended textile; wherein the alkaline hydrothermal treatment comprises a percent change in ΔE (color change) of at least 50%, or 50% to 65%, or 50% to 95%; wherein the hydrothermal treatment is conducted at a pH of pH 7.5 to 11 and at a temperature of 180° C. to 210° C.; wherein the polyester is polyethylene terephthalate; wherein the polyester comprises at least one polyester containing fiber; further comprising providing one or more carbon sources for Halomonas during fermentation; wherein the engineered microbial cell expresses non-native TphA1II; wherein the engineered microbial cell expresses non-native TphA2II; wherein the engineered microbial cell expresses non-native TphA3II; wherein the engineered microbial cell expresses non-native TphBII; wherein the engineered microbial cell expresses non-native TphCII; wherein the engineered microbial cell expresses non-native TphRII; wherein the engineered microbial cell comprises increased activity of at least one or more upstream pathway enzyme(s) leading to improved ethylene glycol utilization, said increased utilization being increased relative to a control cell; wherein the engineered microbial cell is a bacterial cell; wherein the engineered microbial cell is of the genus Halomonas; wherein the engineered microbial cell is of the species elongata; wherein the engineered microbial cell converts glucose and produces at least 50% or 50% to 65% or 50% to 60% of cellular dry weight as PHB; wherein the genome is randomly mutated in genes encoding enzymes selected from the group consisting of alkaline phosphatase, bifunctional allantoicase/(S)-ureidoglycine aminohydrolase, histidine utilization repressor, NO-inducible flavohemoprotein; wherein the alkaline hydrothermal treatment conditions comprise a pH of more than 7.0 and a temperature of at least 120° C.; wherein pH is in the range of 7.5 to 10.5 or 8 to 10; wherein conditions in the fermentation reactor are in the pH range of 7.5 to 10.5 or 8 to 10; a temperature between 15 and 55° C. or between 2° and 50° C., or 30 to 50° C., and optionally a salt concentration (typically NaCl) of between 2 and 25 mass %, or 5 to 25 mass %; wherein hydrothermal treatment occurs at a pH of at least 7.5 and at a temperature of at least 150 or at least 180° C. and optionally up to 300° C. or up to 250° C.; wherein the polymer is an aliphatic polyester; wherein the polyester is poly lactic acid (PLA); wherein the polyester is poly hydroxy butyrate (PHB); wherein the polyester comprises at least one aliphatic polyester fiber; further comprising providing one or more carbon sources for Halomonas during fermentation; wherein the fermentation bioreactor comprises at least 2 g/L lactate and at least 2 g/L crotonate and a lactate/crotonate molar ratio between 0.2 and 5 or between 0.3 and 3.3 or between 0.5 and 2; further comprising at least 2 g/L 3HB and a 3HB/crotonate and/or 3HB/lactate molar ratio between 0.2 and 5 or between 0.3 and 3.3 or between 0.5 and 2; wherein the microbial cell is a bacterial cell; wherein the microbial cell is of the genus Halomonas; wherein the microbial cell is of the species elongata; wherein the microbial cell converts crotonic acid and produces at least 30% or 30 to 65% or 50 to 90% of cellular dry weight as PHB; wherein the microbial cell converts lactic acid and produces at least 30% or 30 to 65% or 50 to 90% of cellular dry weight as PHB; wherein the hydrothermal treatment is conducted at a pH of pH 7.5 to 11 and at a temperature of 180° C. to 210° C.; wherein hydrothermal breakdown products of polyester, biodegradable polymer and food produces at least 15% or 15 to 30% or 20 to 50% of cellular dry weight as PHB.

In another aspect, the invention provides a composition comprising PHB and comprising one or any combination of the following: a cell as described here; at least 2 wt % of PPG; characterizable by any of the properties described herein; 1 to 10 wt % PPG. The invention also includes a composition comprising PHB made by any of the method claims.

Various embodiments contemplated herein include, but are limited to, one or more of the following:

Embodiment 1: An engineered microbial cell that produces 1,6-dihydroxycycloheza-2,4-diene-1,4-dicarboxylate (DHCHDDC), wherein the engineered microbial cell expresses a non-native terephthalate 1,2-dioxygenase (EC 1.14.12.15) comprising tphA1II, tphA2II and tphA3II.

Embodiment 2: The engineered microbial cell of embodiment 1, wherein the engineered microbial cell expresses a non-native1,2-dihydroxy-3,5-cyclohexadiene-1,4-dicarboxylate dehydrogenase (EC 1.3.1.53) tphBII and produces protocatechuate (PCA).

Embodiment 3: The engineered microbial cell of embodiment 2, wherein the engineered microbial cell expresses accessory proteins tphRII and tphC for terephthalate utilization and produces PHB.

Embodiment 4: The engineered microbial cell of embodiment 2, wherein the engineered microbial cell expresses native enzymes to process protocatechuate (PCA) to PHB.

Embodiment 5: The engineered microbial cell of embodiment 4, wherein the engineered microbial cell is of the genus Halomonas, or bacteria having 16s ribosomal RNA-encoding DNA sequence that is at least 80% identical to 16s ribosomal RNA-encoding DNA sequence of Halomonas. Specific strains include Halomonas elongata and their homologs.

Embodiment 6: The engineered microbial cell of embodiment 4, wherein the engineered microbial cell utilizes glucose to produce PHB.

Embodiment 7: The engineered microbial cell of embodiment 4, wherein the engineered microbial cell utilizes ethylene glycol to produce PHB.

Embodiment 8: The engineered microbial cell of embodiment 4, wherein the engineered microbial cell utilizes TPA and ethylene glycol to produce PHB.

In some aspects of embodiment 8, the microbial cell has undergone adaptive evolution.

In some aspects of embodiment 8, the microbial cell genome is randomly mutated in genes encoding enzymes selected from the group consisting of alkaline phosphatase, bifunctional allantoicase/(S)-ureidoglycine aminohydrolase, histidine utilization repressor, NO-inducible flavohemoprotein.

In one embodiment, the invention is directed to a method for degrading polyester-cotton blended textile, comprising contacting the polyester and cotton with one or more engineered microbial cells of Embodiment 8.

Embodiment 9: Microbial cell of the genus Halomonas, or bacteria having 16s ribosomal RNA-encoding DNA sequence that is at least 80% identical to 16s ribosomal RNA-encoding DNA sequence of Halomonas. Specific strains include Halomonas elongata and their homologs.

Embodiment 10: The microbial cell of embodiment 9, wherein the microbial cell utilizes native enzymes to convert acetyl CoA to PHB.

Embodiment 11: The microbial cell of embodiment 10, wherein the microbial cell utilizes native enzymes to convert crotonic acid (CA) to PHB.

Embodiment 12: The microbial cell of embodiment 11, wherein the microbial cell utilizes native enzymes to convert 3-hydroxybutyrate (3HB) to PHB.

Embodiment 13: The microbial cell of embodiment 12, wherein the microbial cell utilizes native enzymes to convert lactic acid (LA) to PHB.

In a further aspect, the microbial cell of embodiment 13 is selected to be salt tolerant from about 100 millimolar to about 3 molar.

In one embodiment, the invention is directed to a method for degrading aliphatic polyester film comprising PLA and/or PHB, comprising hydrothermal treatment of the polyester film and upcycling by contacting the microbial cell in embodiment 13 to generate PHB.

The aliphatic polyester can be a biodegradable polymer. The aliphatic polyester can be mixed with food.

In one embodiment, the invention is directed to a method for degrading a mixture comprising polyester, PLA and food, comprising hydrothermal treatment of the said mixture, and contacting the mixture with one or more engineered microbial cells of Embodiment 8.

In a further aspect, the invention is directed to a method of degrading a mixture comprising polyester, PLA and food and upcycling to generate PHB.

The invention is sometimes described as embodiments. The invention includes separate embodiments as well as any combination of embodiments; the use of the term embodiments is not intended to limit the invention which can be characterized by any combination of embodiments or any features, or portions of embodiments or features, described herein.

The invention includes any of the methods, compositions, schemes, apparatus, systems (apparatus plus fluids and, optionally conditions), embodiments, or data described herein. The method, compositions, system, or apparatus may be further characterized by ±10% or ±20% or ±30% of any of the properties and/or measurements described herein. The invention is further elucidated in the examples below. In some preferred embodiments, the invention may be further characterized by any selected descriptions from the examples or embodiments, for example, within ±30%, ±20% (or within ±10%) of any of the values in any of the examples, tables or figures. “All ranges are inclusive and combinable. For example, when a range of “1 to 5′ is recited, the recited range should be construed as including ranges “1 to 4”, “1 to 3”, “1-2”, “1-2 & 4-5”, “1-3 & 5”, “2-5”, any of 1, 2, 3, 4, or 5 individually, and the like.”

As is standard patent terminology, the term “comprising” means “including” and does not exclude additional components. Any of the inventive aspects described in conjunction with the term “comprising” also include narrower embodiments in which the term “comprising” is replaced by the narrower terms “consisting essentially of” or “consisting of.”

### Glossary

The term “engineered” is used herein, with reference to a cell to indicate that the cell contains at least one targeted genetic alteration introduced by man that distinguishes the engineered cell from the naturally occurring cell.

The term “native” or “wild type” or “WT” is used herein to refer to a cellular component, such as a polynucleotide or polypeptide that is naturally present in a particular cell. A native polynucleotide or polypeptide is endogenous to the cell; that is the term “native” or “wild type” or “WT” refers to sequence characteristics, regardless of whether the molecule is purified from a natural source; expressed recombinantly, followed by purification; or synthesized. The term “native” or “wild type” or “WT” is also used to denote naturally occurring cells.

When used with reference to a polynucleotide or polypeptide, the term “non-native” refers to a polynucleotide or polypeptide that is not naturally present in a particular cell.

The term “host cell” means any type of cell that is susceptible to transformation, transfection, transduction or the like with a nucleic acid construct or expression vector comprising a polynucleotide of the present invention.

The term “heterologous” is used herein to describe a polynucleotide or polypeptide introduced into a host cell. This term encompasses a polynucleotide or polypeptide, respectively, derived from a different organism, species or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promoter and inserted into a different genomic location than that of the native sequence). “Heterologous expression” thus encompasses expression of a sequence that is non-native to the host cell, as well as expression of a sequence that is native to the host cell in a non-native context.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple and/or multiple names, sometimes depending on the source organism from which they derive. The enzyme names used herein encompass orthologs, including enzymes that may have one or more additional functions or a different name.

The term “fermentation” is used herein to refer to a process whereby a microbial cell converts one or more substrate(s) into a desired product (such as PHB) by means of one or more biological conversion steps, without the need for any chemical conversion step.

The term “polyester” encompasses a group of polymers comprising polylactic acid (PLA), polyethylene tetrathalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PESA), polybutylene adipate terephthalate (PEAT), polyethylene furanoate (PEP), polycaprolactone (PCL), poly (ethylene adipate) (PEA) and blends/mixtures of these polymers. Polyester containing material refers to textile or fabrics comprising at least one polyester-containing fiber.

The term “cellulose” as used herein refers to a polysaccharide having the formula (C₆H₁₀O₅)ₙ configured as a linear chain of (1→4) linked D-glucose units. The number of individual glucose monomers in the cellulose polymer defines the degree of polymerization of cellulose.

The term “subcritical water” or “SCW” as used herein refers to liquid water at temperatures between the atmospheric boiling point (100° C.) and the critical temperature (374° C.) that present unique features with respect to its properties, such as density, dielectric constant, ion concentration, diffusivity, and solubility. In the subcritical region, the ionization constant (Kw) of water increases with temperature and is about three orders of magnitude higher than that of ambient water, and the dielectric constant of water drops from 80 to 20.

As used herein, “adaptive evolution” refers to obtaining one or more organisms with one or more desired characteristics through selection for the desired characteristics. It is contemplated that unless explicitly stated otherwise herein, “selection for a desired characteristic” and variations of this root phrase encompasses both positive selection for a desired characteristic and selection against an undesired characteristic. In some embodiments, the selected characteristic comprises at least one of growth rate, consumption of substrate, production rate of PHB, or production efficiently of PHB. In some embodiments, “adaptive evolution” comprises serial passage selection (also referred to as “serial passage exponential” or “SPE” selection. SPE can result in optimization of the strain for target production as well as growth rate. In some embodiments, growth is coupled to target product secretion, and selection is performed for growth. Serial passage selection can comprise passaging cells so that the cells remain in exponential growth phase, and never (or almost never) reach stationary phase. By way of non-limiting example, cells can be transferred when they reach a certain optical density that is characteristic of the cells being in exponential growth phase. In some embodiments, serial passage selection is performed to select for microbial organisms with increased ethylene glycol utilization. In some embodiments, adaptive evolution includes steady state evolution. Steady state evolution can comprise gradually increasing the strength of a selective pressure in order to select for at least one desired characteristic.

### TPA Utilization Pathway

Generally, TPA can be transformed into 1,6-dihydroxycycloheza-2,4-diene-1,4-dicarboxylate (DHCHDDC) under the catalysis of TPA dioxygenase (TphA1II, TphA2II, TphA3II), and DHCHDDC is further oxidized by 1,2-dihydroxy-3,5-cyclohexadiene-1,4-dicarboxylate dehydrogenase (TphBII) to form protocatechuate (PCA). TPA utilization pathway is shown in FIG. 1. Accordingly, a microbial host that utilizes TPA, can be engineered to produce PCA by expressing forms of enzymes TphA1II, TphA2II, TphA3II, TphBII, and accessory proteins TphRII and TphC, that are active in the microbial host. Accessory proteins TphRII and TphC play a role in TPA recognition, folding and initiating the TPA utilization pathway

### Engineering for TPA Utilization in Halomonas.

Any TPA dioxygenase, 1,2-dihydroxy-3,5-cyclohexadiene-1,4-dicarboxylate dehydrogenase, TphRII and TphC that is active in the microbial cell being engineered may be introduced into the cell, typically by introducing and expressing the gene(s) encoding the enzyme(s) s using standard genetic engineering techniques (FIG. 9). Suitable TPA dioxygenase, 1,2-dihydroxy-3,5-cyclohexadiene-1,4-dicarboxylate dehydrogenase, TphRII and TphC may be derived from any source, including plant, archaeal, fungal, gram-positive bacterial, and gram-negative bacterial sources. One or more copies of any of these genes can be introduced into the microbial host cell. In some embodiments, the non-native gene(s) is/are expressed from a strong, constitutive promoter.

### Biosynthesis of Polyhydroxyalkanoates (PHAs)

Polyhydroxyalkanoates are synthesized in a variety of bacterial and archaea genera, including Halobacillus, Bacillus, Salinobacter, Flavobacterium, Chromohalobacter, Halomonas, Marinobacter, Vibrio, Pseudomonas, Halococcus, Halorhabdus, Haladaptatus, Natrialba, Haloterrigena, and Halorussus. As illustrated in FIG. 3, the production of polyhydroxybutyrate (PHB), which is a PHA polymer, involves a three-step enzymatic mechanism that begins with acetyl coenzyme A. The final step of the pathway involves the polymerization of hydroxyalkanoic acid monomers into a hydroxyalkanoate polymer via a polyhydroxyalkanoate polymerase. Bio-synthesized polyhydroxyalkanoates accumulate in the bacterial cell as large molecular weight granules and can account for from about 60% to about 90% of the cellular dry mass.

Ethylene Glycol utilization Pathway

Ethylene glycol is converted enzymatically to glycolate. The engineered microbial cell in this invention uses native enzymes to convert ethylene glycol sequentially into glycoaldehyde, glycolic acid and glyoxylic acid, which is utilized by the cell for cellular growth and PHB production (FIG. 2).

The term “biodegradable” refers to a plastic or polymeric material that will undergo at least partial biodegradation by living organisms (microbes) in anaerobic and aerobic environments (as determined by ASTM D5511), in soil environments (as determined by by ASTM D5988 (, in freshwater environments (as determined by ASTM D5271 (EN 29408)), or in marine environments (as determined by ASTM D6691). The biodegradability of biodegradable plastics can also be determined using ASTM D6868, ASTM D6400, and European EN 13432.

The term “compostable” refers to a biodegradable material that may be broken down into only carbon dioxide, water, inorganic compounds and/or biomass, which does not leave visible or toxic residue.

The term “biomass” refers to an organic or biological material that can be converted into an energy source. One exemplary source of biomass is plant matter. For example, corn, sugar cane, and switchgrass can be used as biomass. Another exemplary source of biomass is crude whole cell product prior to isolation of desired small molecule or polymer. Another non-limiting example of biomass is animal matter, for example cow manure. Biomass also includes waste products from industry, agriculture, forestry, food, perennial grasses, and households. Examples of such waste products that can be used as biomass are fermentation waste, straw, lumber, sewage, garbage and food leftovers. biomass also include sources of carbon, such as carbohydrates (e.g. sugars).

The term “culturing” refers to growing a population of cells, e.g., microbial cells, under suitable conditions for growth, in a liquid or solid medium.

The term “producing” includes both the production of compounds intracellularly and extracellularly, which is to include the secretion of compounds from the cell.

“Carbon source” refers to the types of molecules from which the microorganisms derives the carbon needed for organic biosynthesis.

“ΔE” refers to a change in color between two measurements that can be determined mathematically. For example, a first measurement has coordinates L1*, a1* and b1*, and a second measurement has coordinates L2*, a2* and b2*. The total difference between these two measurements on the CIELAB (Commission Internationale de I-Eclairge) scale can be expressed as ΔE*ab. Generally, if two colors have a ΔE*ab of less than or equal to 1, the difference in color is not perceptible to the human eye. A ΔE*ab of about 2-3 is considered the threshold for perceivable color difference.

### BRIEF DESCRIPTION OF DRAWINGS

The objects, features and advantages of the present invention will be more readily appreciated upon reference to the following disclosure when considered in conjunction with the accompanying drawings.

FIG. 1: Pathway for terephthalic acid (TPA) utilization.

FIG. 2A: Is a heat map representing transcriptomics data indicating log 2 fold change of genes expressed during exponential growth phase of WT H. elongata and Adapted strain 1 using 5 g/L ethylene glycol and 5 g/L glycolic acid as a sole carbon source compared to 5 g/L glucose as a sole carbon source. EG=ethylene glycol, GA=glycolic acid, Glc=glucose. Color blocks on heat map represent the average of at least 2 replicates.

FIG. 2B: Details the pathway map for ethylene glycol utilization in H. elongata.

FIG. 2C: Demonstrates genomic regions of the genes involved in ethylene glycol utilization.

FIG. 3: Overall pathway map illustrating the conversion of waste cotton and polyester (PET) into polyhyroxybutyrate (PHB). Cotton is comprised of glucose (Glc), xylose (Xyl), and arabinose (Ara) polymers that can be converted in various ways into monomeric units which can then converted to intracellular intermediates such as phosphoenolpyruvate (PEP) and acetyl COA (AcCoA) which can be used for the production of PHB. Similarly, ethylene glycol (EG) and terephthalic acid (TPA) can be converted to intracellular intermediate such as protocatechuic acid (PCA), acetyl COA (AcCoA), succinyl CoA (SucCoA), succinate (Suc), oxaloacetate (OAA), and citrate (CIT), all of which can also be used to produce PHB. Expanded names of enzymes and intermediates are listed on the right.

FIG. 4: Illustrates growth of the adapted engineered H. elongata strain demonstrating growth in the presence of PLA/plastic, PLA/plastic containing peanut butter and peanut butter only conditions. Higher optical densities (OD) obtained in PLA/plastic and PLA/plastic plus peanut butter conditions, demonstrate robust growth under these conditions.

FIG. 5A: Graphical diagram illustrating the TPA yield and percent residual solids resulting from alkaline hydrothermal treatment of dyed, mixed (1:1 mixture of cotton and polyester) textile waste, compared to undyed and separate cotton and polyester textile.

FIG. 5B: Provides quantification of TPA yield and % residual solids corresponding to FIG. 5A. Values are averages of three independent experiments, +SD.

FIG. 6: ¹H NMR of precipitated TPA from the selective hydrothermal breakdown of black dyed polyester t-shirt with black dyed white t-shirt. Commercial TPA is shown for reference.

FIG. 7: Growth of WT Halomonas elongata using different sources in MM63 medium. Glc=glucose, EG=ethylene glycol, SCW=subcritical water product 55918-1, no C=no carbon control. MM63 medium contained 60 g/L NaCl set at pH=8.0. Points represent the average of three replicates and error bars are one standard deviation. Note that the SCW water products were estimated to be ˜1.6 g/L ethylene glycol based on NMR data, so we observe high conversion of ethylene glycol to biomass.

FIG. 8: Adaptive evolution was performed by passaging cells serially using 5 g/L ethylene glycol as a sole carbon source. This growth indicates that the selection process introduced mutations into the genome that enable robust growth of H. elongata on ethylene glycol as a sole carbon source. The plot shows the growth of two isolates purified from adaptive laboratory evolution (Adapted 1 and Adapted 2) compared to the WT strain.

FIG. 9: Workflow for H. elongata engineering for plasmid transformation by conjugation.

FIG. 10: plasmid map indicating genes introduced to the H. elongata genome by a replicated plasmid to enable terephthalic acid utilization.

FIG. 11A: Shows the growth of the WT strain (or parent strain), H. elongata DSMZ 2581 on glucose (Glu) and ethylene glycol (EG). We observe slow growth on ethylene glycol as a sole carbon source.

FIG. 11B: Shows the growth of Adapted strain 1 on glucose and ethylene glycol, where we see a reduced lag phase and higher optical density (OD600).

FIG. 12A: Is a graphical illustration of the growth of the WT strain (or parent strain), H. elongata DSMZ 2581 on glucose (Glu) and terephthalic acid (TPA). We do not observe growth on terephthalic acid as a sole carbon source.

FIG. 12B: Illustrates the growth of engineered H. elongata on TPA as a sole carbon source while also retaining ability to consume glucose as a carbon source, and growth is relatively unaffected. For plots, points represent the average of three replicates and error bars show one standard deviation.

FIG. 13A: Is a graphical illustration of the growth of the WT strain (or parent strain), H. elongata on glucose, ethylene glycol (EG), terephthalic acid (TPA), and mixtures thereof.

FIG. 13B: Illustrates growth of the engineered H. elongata strain demonstrating growth using terephthalic acid and improved utilization of ethylene glycol compared to the WT strain, and higher optical densities reached, demonstrating conversion of multiple substrates into biomass and PHAs.

FIG. 14A: Is a flow chart demonstrating how textile waste material is processed. We treat cotton and other end of life textiles with a hydrothermal treatment to release polyester monomers and other compounds present in the textile, such as dyes, leaving cotton material as a by-product. Then, we treat the cotton by-product with enzymes that can break the glycosidic bonds present in the cotton material so that glucose is released.

FIG. 14B: Is a graphical plot where glucose release was quantified by mass loss of the material after cellulase treatment. The released glucose/sugar stream was fed to H. elongata for PHB production. Bars represent the average of three replicates and error bars show one standard deviation.

FIG. 15: Growth of H. elongata in culture medium containing 5.4 g/L glucose.

FIG. 16A: Demonstrates the growth of engineered H. elongata using mock cotton t-shirt products and PHB production reaching ˜60% cell dry weight (CDW) polyhydroxybutyrate (PHB) content.

FIG. 16B: Shows a photo of PHB material after extraction and quantification.

FIG. 17: Growth of engineered H. elongata using t-shirt breakdown products from the hydrothermal treatment for PHB production.

FIG. 18: UV-vis spectra of dyed and undyed (white) textiles.

FIG. 19A: Residual solids of cotton textile samples after hydrothermal treatment at varied temperature.

FIG. 19B: images of aqueous and solid fractions after hydrothermal treatment of cotton samples at varied temperature.

FIG. 20: ATR-IR spectra comparison for untreated cotton and hydrothermally treated at varied temperature.

FIG. 21: ¹H NMR comparison of Battelle's PHB sample and commercial PHB from Sigma Aldrich (Product No: 363502). Peaks for PHB and the PPG contaminated are indicated.

FIG. 22: ¹H NMR comparison of the large scale PHB sample across 3 different regions. This data indicated near complete uniformity across the entire sample with regards to the ratio of PHB and PPG.

FIG. 23: ¹H NMR spectrum showing purified Battelle PHB and commercial PHB.

FIG. 24: GPC chromatogram for Battelle PHB pre-extraction of PPG (red) and commercial PHB (purple).

FIG. 25: Mn and Mw values of Battelle large scale PHB material, commercial PHB (Sigma Aldrich, product no. 363502), and a 500 kDa Mw PHB standard (Sigma Aldrich, product no. 915092). Error bars represent the standard deviation across two GPC runs of the same sample.

FIG. 26: Flow diagram showing exemplary steps of a process for mixed textile deconstruction and upcycling to PHAs according to the present disclosure.

FIG. 27: Overall pathway map illustrating the destruction of polylactic acid (PLA) and polyhydroxybutyrate (PHB) and synthesis of PHB for circularity according to the present disclosure. Hydrothermal treatment of PLA substrate generates lactic acid (LA) that is metabolized by native enzymes in the microbe to yield PHB. Hydrothermal treatment of PHB substrate generates 3-hydroxybutyrate (3HB) and crotonic acid (CA). 3HB and CA are metabolized by microbial enzymes to generate PHB. Expanded names of enzymes and intermediates are listed on the right.

FIG. 28A: Hydrothermal deconstruction of PLA and PHB. Table illustrating hydrothermal depolymerization reaction conditions for PLA and PHB substrates.

FIG. 28B: Images demonstrate materials required and reaction products of the hydrothermal reaction. Table demonstrating the weight of residual solids from reaction corresponding to FIGS. 28A&B and percent depolymerization.

FIG. 29A: Monomers contained in the reaction products. Quantification of monomers present in the reaction products as described in FIG. 28, determined using LC/MS.

FIG. 29B: Expected and actual concentrations and yield of monomers from solid based on reactor loading and dilution for LC/MS measurement. Replicates represent technical replicates from a representative reactor run.

FIG. 30A: Schema of PLA and PHB deconstruction and utilization of reaction products by biological transformation.

FIG. 30B: Growth of Halomonas elongata DSMZ 2581 in MM63 medium containing 3 g/L lactate, 3 g/L crotonate, and mixture of 3 g/L lactate and 3 g/L crotonate as indicated. MM63 medium contained 13.61 g/L KH·PO₄, 4.21 g/L KOH, 1.98 (NH₄)₂SO₄, 0.0011 g/L FeSO₄—H2O 0.25 g/L MgSO₄-7H₂O, and 60 g/L NaCl set at pH=8.0. Points represent the average of three replicates and error bars are one standard deviation.

FIG. 31A: Upcycling and PHB yield. Growth of Halomonas elongata in MM63 medium containing deconstruction products from PLA and PHB resin. Plot indicates improved utilization of reaction products and higher optical densities reached, demonstrating conversion of reaction products into polyhydroxyalkanoates. MM63 medium contained 60 g/L NaCl set at pH=8.0.

FIG. 31B: Measured concentrations of deconstructed products from PLA/PHB mixture during growth with multiple additions of concentrated deconstruction products from PLA and PHB resin with measurement of concentrations done using LC/MS over course of experiment. After addition of more deconstruction products, an additional measurement of deconstruction product concentrations was performed, yielding a sawtooth shaped curve.

FIG. 31C: Measurements to determine % CDW of PHB from deconstruction products.

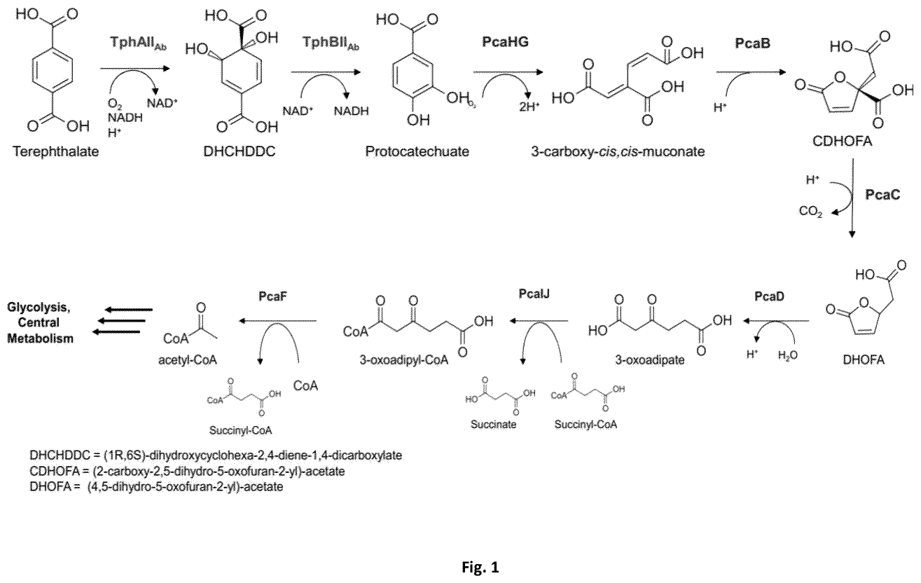

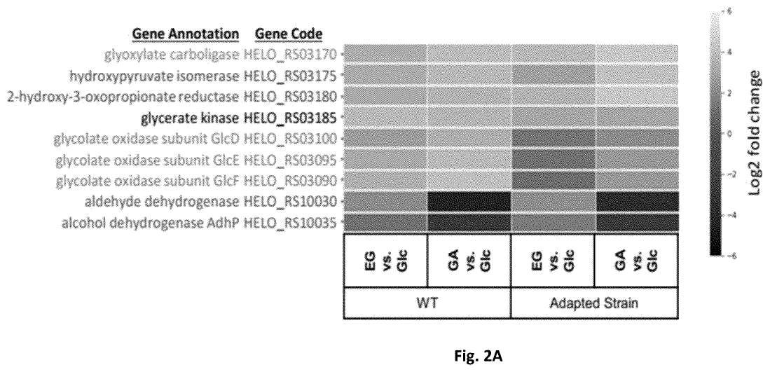

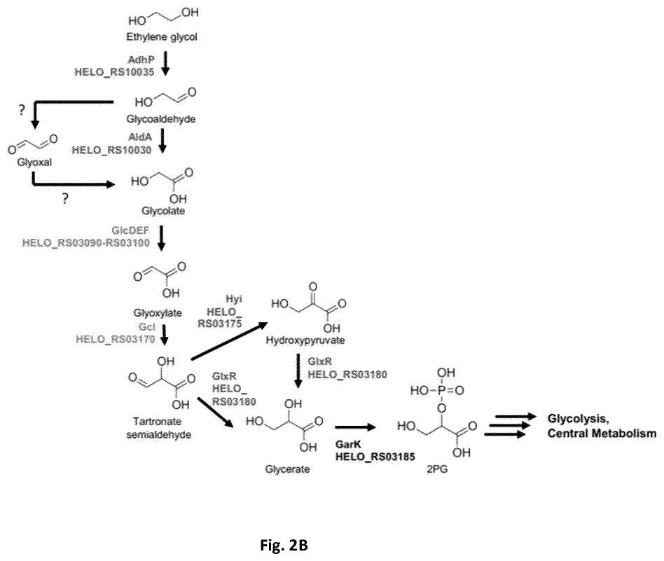

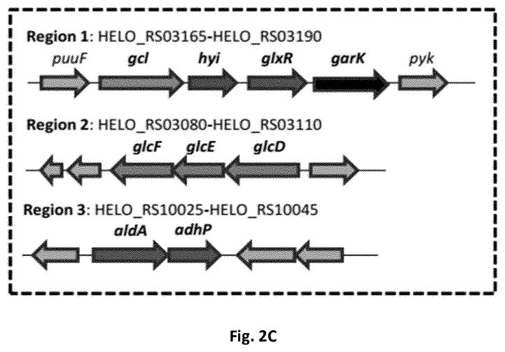

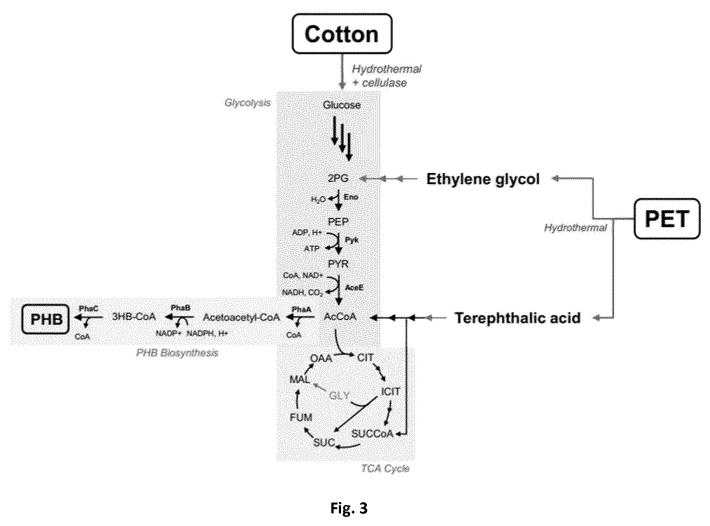

### DETAILED DESCRIPTION OF THE INVENTION

The invention provides methods for the deconstruction and upcycling of mixed polyester-cotton blended textile waste via fermentation by a microbial host from carbon and nitrogen sources such as glucose, terephthalic acid (TPA) and ethylene glycol. The aim was achieved via the introduction of a non-native TPA utilization pathway into a suitable microbial host for industrial fermentation of large-scale chemical products. The engineered TPA utilization pathway links the central metabolism of the host to the non-native pathway to enable the production of polyhydroxybutyrate (PHB). In another aspect the disclosure describes a method and system for the deconstruction and upcycling of biodegradable plastic PLA and PHB via hydrothermal depolymerization and fermentation by a microbial host. The aim was achieved via generation of reaction products from hydrothermal depolymerization that provides the carbon source for a suitable microbial host for fermentation and generation of PHB. In an additional aspect, the disclosure describes a method and system for the hydrothermal deconstruction and microbial upcycling of biodegradable PLA, polyester and food. The reaction products are suitable substrates that biologically transform in the microbial host to enable production of PHB.

The present invention provides an engineered Halomonas strain that expresses non-native enzymes TphA1II, TphA2II, TphA3II, TphBII, and accessory proteins TphRII and TphC.

In some embodiments, the present disclosure provides an engineered Halomonas strain that expresses randomly mutated sequences as in SEQ ID 1, 2, 3, 4 (table 1). Here the following nomenclature is used: Original nucleotide position, mutation to substituted amino acid and gene containing the mutation. Accordingly, for example in SEQ ID 1, the mutation of GAG to GAT at nucleotide position 645,660 is designated as amino acid mutation G->T.

In one embodiment, the present invention provides the Halomonas strain that expresses native enzymes that can metabolize reaction products lactic acid (LA), crotonic acid (CA), 3HB.

Substrates or intermediates, for example acetyl-CoA, lactic acid, crotonic acid, 3HB may be diverted to the synthesis of PHB. In some non-limiting embodiments, some fraction of carbon flux along the various biosynthesis pathways is directed into the biosynthesis of PHB.

Vectors are tools used to shuttle DNA between host cells or as a means to express a polynucleotide sequence. Inserting the DNA of interest, such as the sequences for TphA1II, TphA2II, TphA3II, TphBII, TphRII and TphC is accomplished by ligation techniques and/or mating protocols well known to the skilled artisan. Such DNA is inserted such that its integration does not disrupt any necessary components of the vector. In the case of vectors that are used to express the inserted DNA as a polypeptide, the introduced DNA is operably-linked to the vector elements that govern its transcription and translation. Vectors have many manifestations, A “plasmid” is a circular double stranded DNA molecule that can accept additional DNA fragments. Certain vectors are capable of autonomous replication is a host cell (e.g., bacterial vectors having a bacterial origin of replication and episomal mammalian vectors). Vector choice is dictated by the organisms or cells being used and the desired fate of the vector. Vectors can replicate once in the target cell, or can be “suicide” vectors. In general, vectors comprise signal sequences, origins of replication, marker genes, enhancer elements, promoter, and transcription termination sequences. Vectors often use a selectable marker to facilitate identifying those cells that have incorporated the vector.

The alkaline hydrothermal approach for textile deconstruction is designed to accommodate mixed polyester-cotton waste. Textile deconstruction depolymerizes synthetic fibers, strips and solubilizes dyes, and the cellulase treatment deconstructs cotton fibers. The hydrothermal treatment is energy efficient, occurs at mild conditions (180-200° C.) and is efficient with a short residence time (15-30 min). The alkaline hydrothermal treatment, which requires salt, is compatible for fermentation by Halophilic strains that thrive in high pH and briny media.

In a method of this invention, the hydrothermal approach for aliphatic polyester deconstruction is designed to accommodate PLA and PHB. Hydrothermal deconstruction depolymerizes aliphatic polyesters PLA and PHB resulting in reaction products crotonic acid, lactic acid and 3HB. The hydrothermal treatment is energy efficient, occurs at mild conditions (180° C.) and has a short residence time (30 min) to limit processing costs. The alkaline hydrothermal treatment, which requires salt, is compatible for fermentation by halophilic strains that thrive in high pH and briny media. This unique combination of characteristics renders Halomonas as a suitable host microbial cell.

A bioreactor or fermenter is used to culture microbial cells through the various phases of their physiological cycle. A bioreactor is utilized for the cultivation of cells, which may be maintained at particular phases in their growth curve. The use of bioreactors is advantageous in many ways for cultivating optimal growth. Generally, the control of growth conditions including control of dissolved carbon dioxide, oxygen and other gases, dissolved nutrients, trace elements, temperature and pH is facilitated in a bioreactor.

Process conditions in the bioreactor are used to enhance the effect on biosynthesis of native enzymes of microbial cells. In some embodiments, the process conditions used to enhance the effect on the native enzymes may include temperature and pH. The pH of the microbial culture may be controlled. In certain embodiments pH is controlled within an optimal range for microbial maintenance and/or growth and/or conversion of feedstock and/or production of PHB and/or survival.

The microorganism of the present invention can accumulate PHB to over 30% and/or over 40% and/or over 50% of the total cell mass. In some non-limiting embodiments, the microorganism is Halomonas elongata DSMZ 2581.

To give an illustration of the application of a bioreactor/fermenter in certain embodiments of the present invention, a bioreactor containing nutrient medium is inoculated with the microorganism. Generally, there will follow a lag phase prior to cells beginning to double. After the lag phase, the cell doubling time decreases and the culture goes into the logarithmic phase. The logarithmic phase is eventually followed by an increase of the doubling time that, while not intending to be limited by theory, is thought to result from either a mass transfer limitation, depletion of nutrients including nitrogen or mineral sources, or a rise in the concentration of inhibitory chemicals, or quorum sensing by the microbes. The growth slows down and ceases when the culture goes into the stationary phase. In order to harvest cell mass, the culture in certain embodiments is harvested in the logarithmic phase and/or the arithmetic phase and/or in the stationary phase.

In some cases, the bioreactor at inoculation is filled with a starting batch of nutrient media and/or additives at the beginning of growth and no additional nutrient media and/or additives are added after inoculation.

Inoculation of the culture into the bioreactor may be performed by methods including, but not limited to, transfer of culture from an existing culture inhabiting another bioreactor, or incubation from a seed stock raised in an incubator. The seed stock of the strain may be transported and stored in forms including a powder, liquid, frozen, or freeze-dried form as well as any other suitable form, which may be recognized by one skilled in the art. In certain non-limiting embodiments, the reserve bacterial cultures are kept in a metabolically inactive, freeze-dried state until required for restart. When establishing a culture in a very large reactor, cultures can be grown and established in progressively larger intermediate scale vessels prior to inoculation of the full-scale vessel.

The bioreactors may have mechanisms to enable mixing of the nutrient media that include, but are not limited to, one or more of the following: spinning stir bars, blades, impellers, or turbines: spinning, rocking, or turning vessels; gas lifts, sparging;

recirculation of broth from the bottom of the container to the top via a recirculation conduit, flowing the broth through a loop and/or static mixers. The culture media may be mixed continuously or intermittently.

The method of this invention may be practiced with the Halomonas strains disclosed herein or with microorganisms that have substantially the same genetic background and phenotypes. Examples of such microorganisms include those that have an rDNA nucleotide sequence that is 80% or more identical, e.g., 80, 81, 82, 83, 84, 85, 86, 87, 88, 89, 90, 91, 92, 93, 94, 95, 96, 97, 98, 99, <100 or 100% identical to a corresponding sequence of Halomonas strains disclosed herein. BLAST may be used to determine the percentage identity or similarity of rDNA of a strain to that of the Halomonas strains disclosed herein. BLASTN may be used to identify a polynucleotide sequence having at least 70%, 75%, 80%, 85%, 87.5%, 90%, 92.5%, 95%, 97.5%, 98%, 99% sequence identity to a reference polynucleotide. A representative BLASTN setting optimized to find highly similar sequences uses as Expect Threshold of 10 and a Wordsize of 28, max matches in a query range of 0, match/mismatch scores of 1/−2, and linear gap cost. Low complexity regions may be filtered or masked.

The invention specifically contemplates new strains of the Halomonas strains disclosed herein including subcultures, mutational variants and genetically engineered variants. Mutation and genetic engineering of bacteria is well known in the art. Mutational and genetically engineered variants will include at least one variation to a gene or genomic sequence of a parental strain. Preferably, such variants will retain or enhance or otherwise modulate the capacity of the parental strain to metabolize ethylene glycol and TPA, or increase or decrease its growth at particular temperatures, pH or under altered conditions of salinity or in the presence of other compounds or competing microorganisms. Such variants preferably have at least 70, 80, 90, 95, 96, 97, 98, 99, <100% sequence identity or similarity to partial or whole rDNA sequences or to entire chromosomal sequences of the strains disclosed herein.

Variants, such as epigenetic variants or other types of non-genetic or phenotypic variants, are also contemplated. These may be produced by serial passage and selection of strains disclosed herein in a medium that selects for growth and viability at a particular temperature, pH and salinity. Such variants will generally exhibit at least one phenotypic variation compared to a parent strain. For example, their growth rate may be 1, 2, 3, 4, 5, 6, 7, 8, 9, 10, 15, 20, 50 or 100% or any intermediate value) higher under the same culture conditions or they may have the same or higher growth rate than a parent strain when pH, temperature or salinity is varied (increased or decreased) by 1, 5, 10, 25, 50 or 100% (or any intermediate value) of an optimal value for a parent strain. For example, a Halomonas variant may exhibit the same or higher growth rate at a pH±0.25, 0.5, 1, 1.5, 2.0, 2.5 or 3.0 different than the optimal pH for growth or ability of the parental strain to utilize EG and/or TPA, may exhibit the same or higher growth rate at a temperature±1, 2, 3, 4, 5, 6, 7, 8, 9, or 10° C. different than the optimal temperature for growth of a parental strain or for its ability to utilize EG and/or TPA, or may exhibit the same or higher growth rate when the salinity is increased or decreased by 1, 2, 3, 4, 5, 6, 7, 8, 9, 10 wt % compared to the optimal salinity for growth of the parental strain or for the ability to utilize EG and/or TPA.

TABLE 1
Mutations detected in Halomonas strains with improved
ethylene glycol utilization obtained by adaptive evolution

| SEQ ID | Position | Mutation | Annotation | Gene | Description |
|---|---|---|---|---|---|
| 1 | 645,660 | G→T | E200D (GAG- | HELO_RS02960 | Alkaline |
| | | | GAT) | | phosphatase |
| 2 | 690,425 | G→A | C251Y (TGC- | HELO_RS03165 | Bifunctional |
| | | | TAC) | | allantoicase/(S)- |
| | | | | | ureigoglycine |
| | | | | | aminohydrolase |
| 3 | 1,721,568 | C→T | E141E (GAG- | hutC | Histidine utilization |
| | | | GAA) | | repressor |
| 4 | 2,211,050 | G→A | G336E (GGG- | hmpA | NO-inducible |
| | | | GAG) | | flavohemoprotein |

### Example 1: Hydrothermal Depolymerization of Textile

The following is a set of reaction conditions that can be generalizable for the co-hydrothermal treatment of cotton and polyester t-shirts:

A 1:1 mixture of cotton and polyester t-shirts (usually 2.5 g each) was submerged in 50 mL H₂O in a stainless steel-autoclave. Optionally, an acidic (HCl, acetic acid, formic acid) or a basic (NaOH) additive was used to adjust the pH of the reaction solution. The sealed autoclave was heated to reaction temperature (180-250° C.) and held for the duration of the reaction (15-60 minutes). Upon completion of the reaction, the autoclave was rapidly cooled to room temperature. The reaction contents were removed from the autoclave and were filtered using vacuum filtration. The collected residual solids were washed with H₂O and dried in an oven at 80° C. for 2 h to remove any residual water. The mass of the residual solids was measured and recorded. The filtrate, commonly consisting of dissolved sodium terephthalate and ethylene glycol, was acidified to pH=1 via the addition of HCl, precipitating out terephthalic acid (TPA) as a white powder. The TPA was isolated via vacuum filtration, and the collected TPA was washed with H₂O and dried in an oven at 80° C. for 2 h to remove any residual water. The mass of the dried TPA was measured and recorded.

### Example 2: Hydrothermal Breakdown of White Polyester with Cotton T-shirts

A 1:1 mixture of undyed polyester/cotton t-shirt was reacted in 50 ml of water with various acid or base catalysts described for other hydrothermal reactions in the literature. Overall, all runs at 250° C. and all acid conditions tested resulted in unacceptably high charring of the t-shirt material. Thus, the reaction temperature was reduced to 200° C. and conducted in basic conditions using 0.5M NaOH as an additive. These reaction conditions resulted in what appeared to be the selective depolymerization of polyester to its monomers, ethylene glycol and sodium terephthalate. Some cotton discoloration occurred under these conditions, which is presumed to be charring, but the cotton otherwise appeared to not be substantially altered. Lowering the reaction temperature to 180° C. appeared to decrease the extent of discoloration, while still fully breaking down the polyester t-shirt. With these reaction conditions, TPA was isolated as a white powder (1.78 g, 82.4% yield). It was concluded from this that the set of conditions that were desirable for selectively depolymerizing white polyester t-shirts while unaffecting white cotton t-shirts were as follows:
Mass of t-shirts: 2.5 g each polyester and cotton t-shirts
Solvent: 50 mL H₂O
Additive: 1 g NaOH
Reaction temperature: 180-200° C.
Reaction duration: 15-30 minutes

### Example 3: Hydrothermal Breakdown of Black Dyed Polyester with Cotton T-Shirts

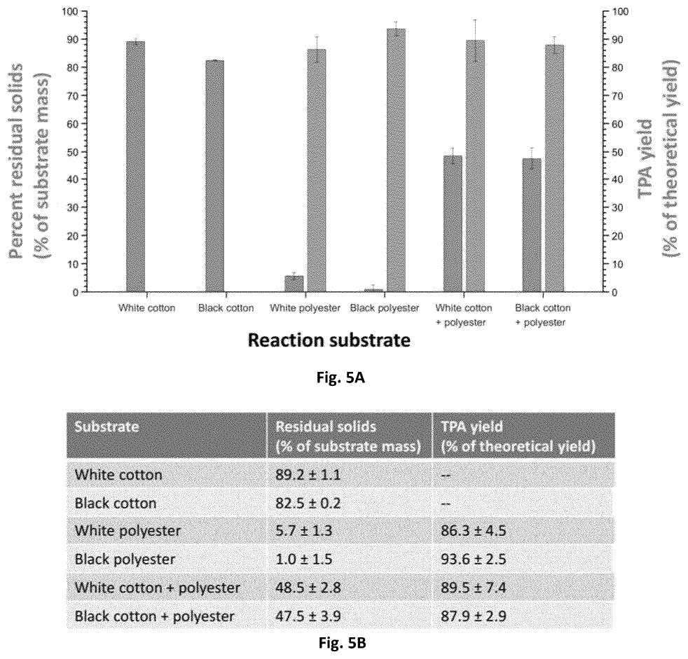

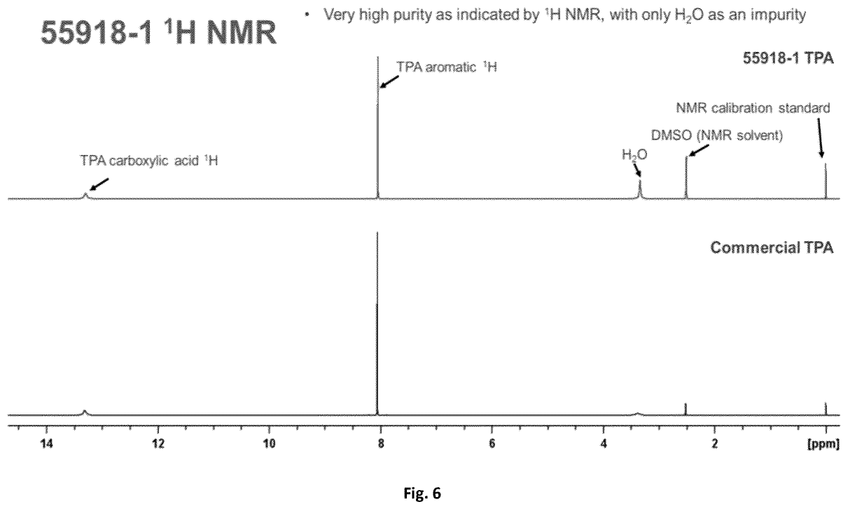

Following these promising results for white t-shirts, the same conditions were then tested for dyed black t-shirts to determine the effect of the dyes on the hydrothermal breakdown process. After the co-treatment of both black cotton and polyester t-shirts under these optimized conditions, visually only the cotton t-shirt remained. While there was minimal physical/mechanical change to the fibers of the cotton t-shirt and low mass loss (2.16 g residual solids), there was significant quantities of the dye that were removed from the cotton and dissolved in solution. The resulting cotton t-shirt was light brown in color, with a dark blue aqueous fraction. There was a TPA yield of at least 82.4%, calculated using a theoretical yield that does not factor in the mass of the dye. Because the dyes constitute some of the total mass of the t-shirt and there is mass loss during the workup process, the actual yield likely even higher. Although the isolated TPA was a light purple color, discoloration is expected when dyes are involved in processing conditions. Despite this, ¹H NMR indicated a high level of purity of the produced TPA, with no detectible impurities beyond trace H₂O being present (FIG. 6).

Additionally, both cotton and polyester black t-shirts were tested separately as controls under the same reaction conditions. The black cotton t-shirt yielded 2.06 g residual solids (17.6% mass loss) and a dark blue aqueous fraction, demonstrating that while the cotton fibers remain mechanically intact during these reaction conditions, there is some mass loss either due to dye leaching or minor thermal decomposition of cotton. The black polyester shirt yielded one percent residual solids, 93.6% yield of TPA, and a dark blue aqueous fraction, demonstrating a near-complete breakdown of polyester. These results are also graphically summarized in FIG. 5. Like for the white t-shirts, these conditions similarly proved to be able to selectively break down black polyester t-shirts while leaving black cotton t-shirts intact. These results are significant in that they demonstrate that the presence of dyes does not have a significant effect on the selective hydrothermal breakdown of t-shirts, potentially allowing for the simultaneous breakdown of t-shirts containing a variety of different dyes.

### Example 4: Scaling of Selective Hydrothermal Breakdown of Polyester with Cotton T-Shirts

Following successful demonstration of selective polyester deconstruction in a polyester: cotton blend of dyed textile waste, a scaling of this reaction was performed. 25 g of black-dyed polyester shirt and 25 g of black-dyed cotton shirt material were reacted with 350 g of deionized water and 10 g of NaOH. 92% yield (19.8 g) of the theoretical yield of TPA was recovered, with 25.8 g of residual solids, being primarily cotton. Visually, the cotton material was significantly stripped of black dye as previously observed at 2.5 g scale reactions.

### Example 5: Halomonas Utilization of Subcritical Water (SCW; i.e. Alkaline Hydrothermal Treatment) Breakdown Products

We also explored the growth of Halomonas elongata on subcritical water products. We selected SCW product 55918-1, which contained both polyester post consumer textile with a black dye. These were deconstructed to monomers (terephthalic acid and ethylene glycol) and the terephthalic acid was precipitated out for characterization, leaving a remaining ethylene glycol waste stream. This was prepared with minimal salts medium directly and used as a growth substrate for the WT Halomonas elongata strain.

The SCW product contained 4.1 g/L EG, and the final medium preparation contained ˜1.6 g/L EG. We reached a final optical density of 1.08 after 48 hours, compared to a final OD600 of 3.16 for 5 g/L ethylene glycol. Assuming linearity in biomass to substrate loading, and assuming the major carbon source was ethylene glycol, we would expect to see a final OD600 of 1.04 for our ethylene glycol waste stream, which suggests that we had high conversion of ethylene glycol (FIG. 7).

### Cell Density:

Cell density was measured using a spectrophotometric assay detecting absorbance of each well/cuvette at 600 nm. The measurements were performed using a Thermo Scientific Genesys 150 spectrophotometer. A non-inoculated control was used to subtract background absorbance.

Minimal salts medium: 13.61 g/L KH·PO₄, 4.21 g/L KOH, 1.98 (NH₄)₂SO₄, 0.0011 g/L FeSO₄—H2O 0.25 g/L MgSO₄-7H2O, 60 g/L NaCl.

### Example 6: Saccharification of Cotton by Ctec2 Cellulase Mix and Growth of WT Halomonas elongata

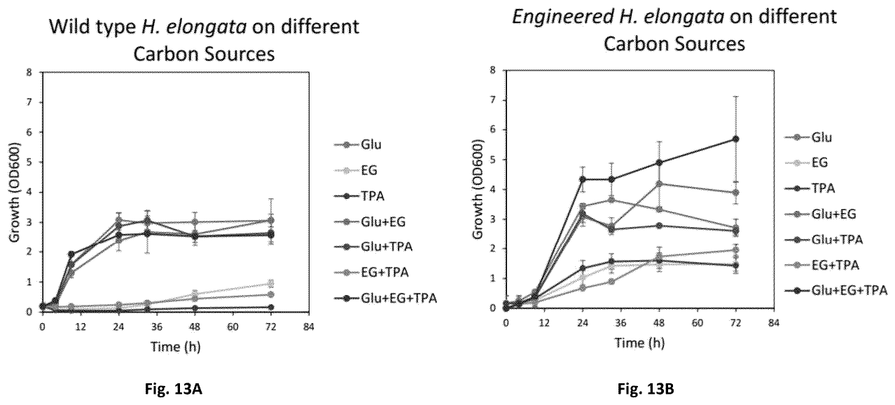

We investigated the breakdown of hydrothermally treated cotton by product (FIG. 14) from the reaction to determine if saccharification was possible. We took a 1 g swatch of hydrothermally treated textile and subjected the cotton material to the Ctec2 cellulase treatment at a pH of 5, temperature of 50° C., and a loading of 30 FPU/g material. We observed an 18% mass loss after 72 hours of treatment, indicating that the cotton material is digestible by commercial cellulase mixes and further optimization by cellulases is possible. This cotton breakdown material can also be fed for growth and upcycling of the waste stream to polyhydroxybutyrate or other polymers.

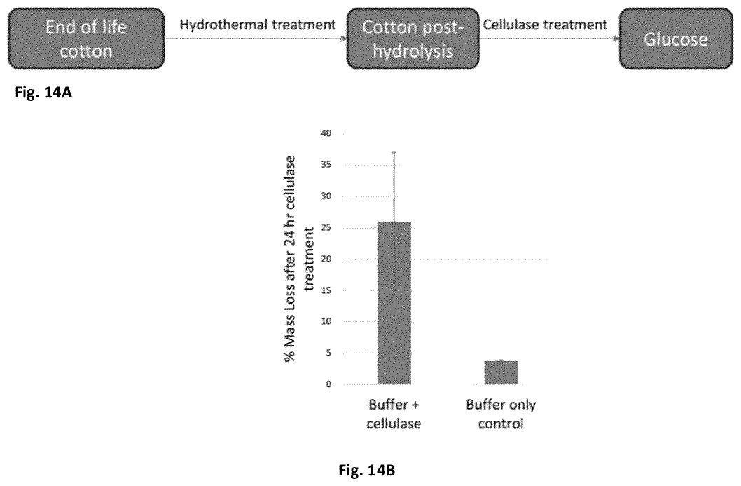

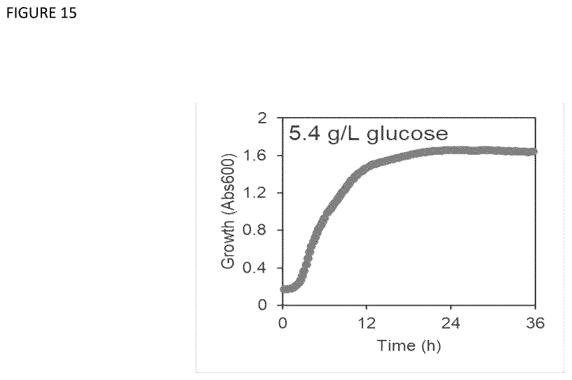

Ctec2 cellulases are well known to depolymerize cotton into glucose or other sugar isomers. FIG. 15 shows that WT Halomonas elongata can consume glucose and grow rapidly, as expected.

### Example 7: Engineering Halomonas elongata for TPA Utilization and Improved EG Utilization

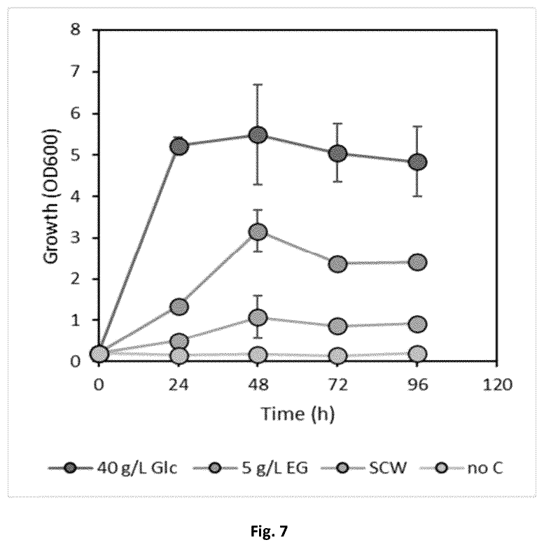

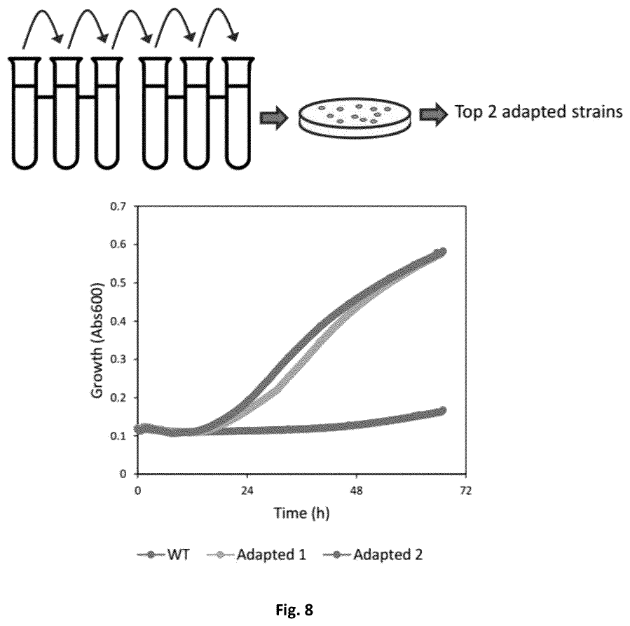

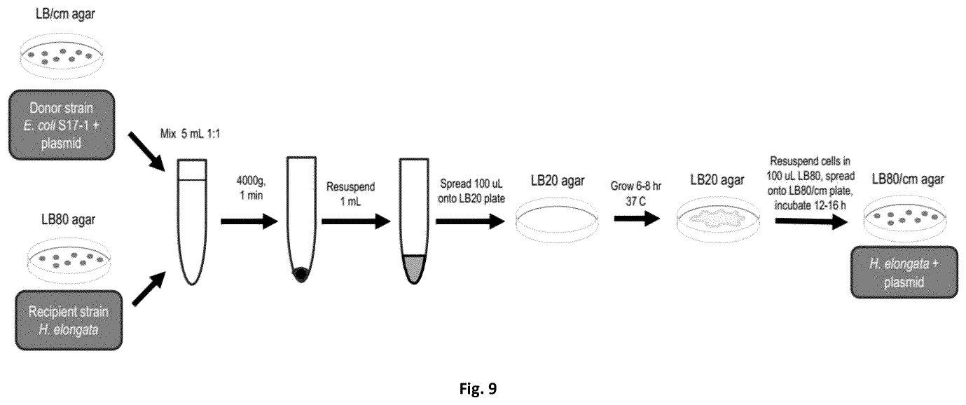

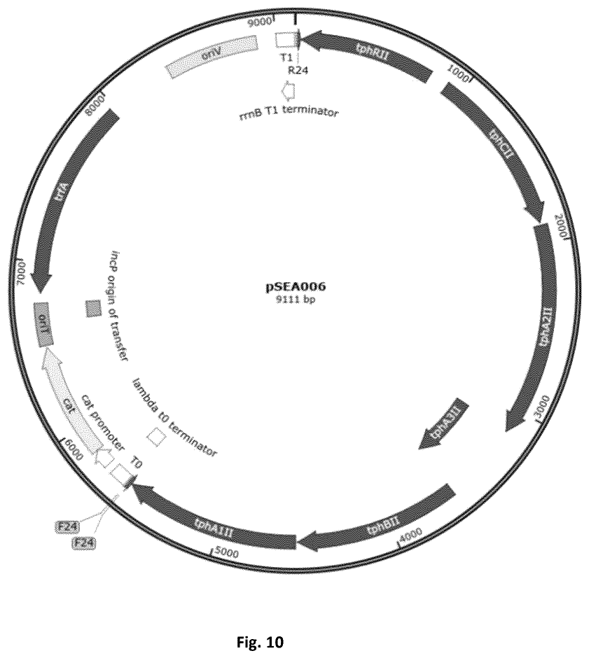

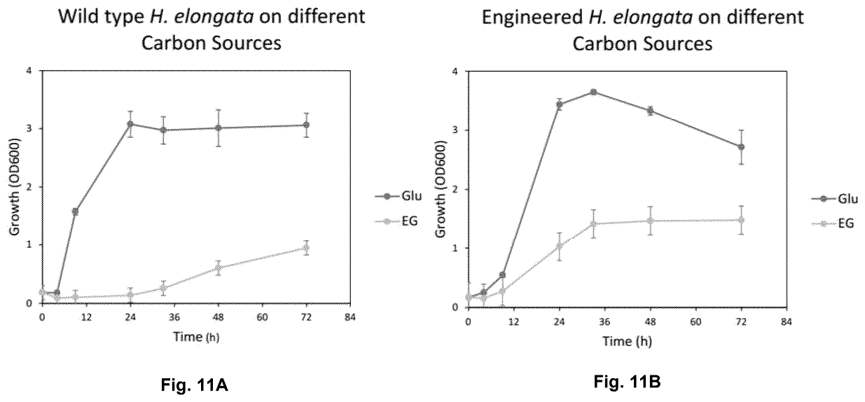

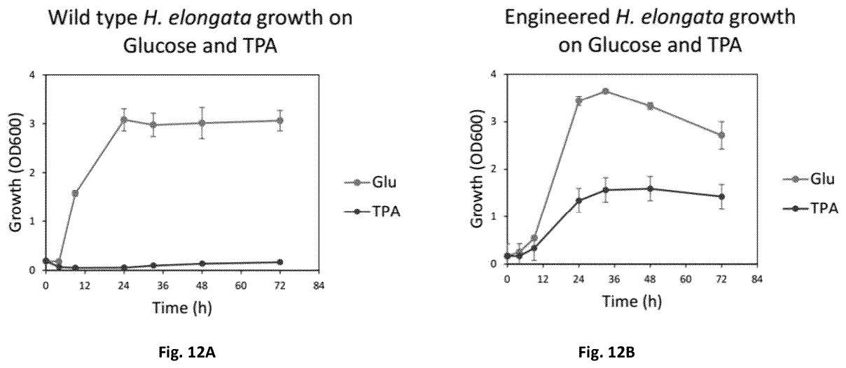

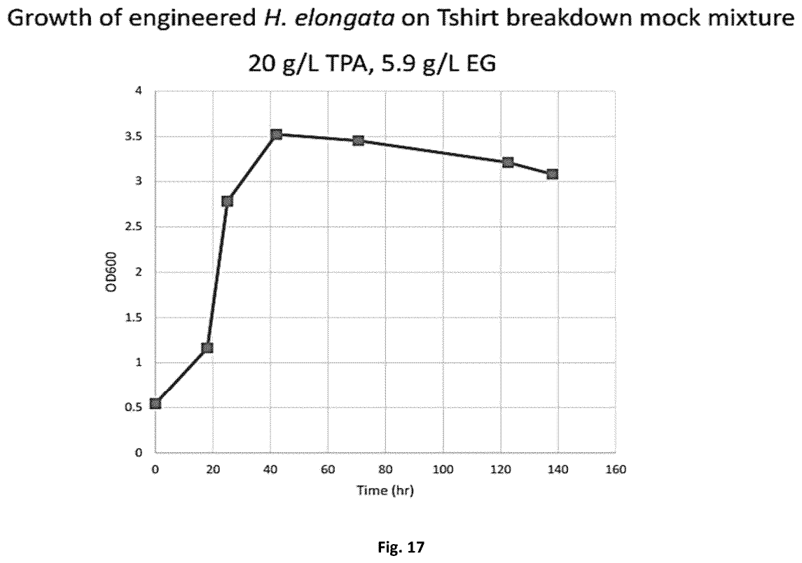

To improve utilization of EG, WT Halomonas elongata strain, was serially passaged (FIG. 8) using 5 g/L ethylene glycol as a sole carbon source in the culture medium. Serial passaging allowed the selection of the top two adapted strains with robust growth on ethylene glycol as a sole carbon source. The adapted strain (adapted 1) of Halomonas elongata was subsequently engineered for TPA utilization using standard genetic engineering techniques well-known in the art. Briefly, the genes for Terephthalate 1,2-dioxygenase (EC 1.14.12.15, TphA1II, TphA2II, TphA3II), 1,2-dihydroxy-3,5-cyclohexadiene-1,4-dicarboxylate dehydrogenase (EC 1.3.1.53, TphBII), and corresponding DNA response regulator and receptor, the accessory proteins TphRII and TphC from Acinetobacter baylyli ADP1 were inserted to the replicative plasmid pSEVA321 to obtain the recombinant plasmid vector pSEA006 (FIG. 10). The recombinant plasmid was transformed into the E. coli donor strain S17-1. As shown in FIG. 9, the adapted strain (adapted 1) of Halomonas elongata was combined with the transformed E. coli donor strain to obtain engineered Halomonas elongata containing the recombinant plasmid vector pSEA006. As illustrated in FIGS. 11-13 and 17, the engineered Halomonas elongata demonstrated improved utilization of EG, TPA and t-shirt breakdown mock mixtures.

### Example 8: Characterization of PHB

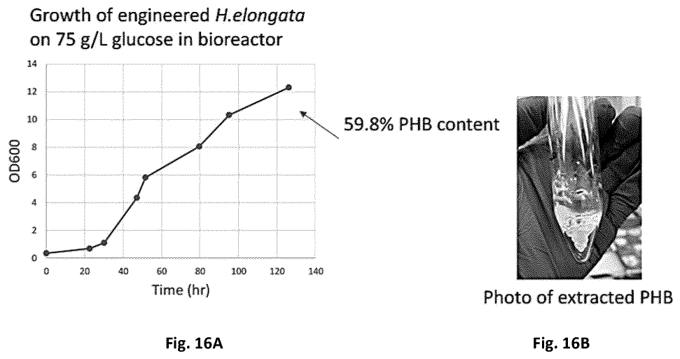

As shown in FIG. 16, the polyhydroxybutyrate polymer was derivatized into methyl-3-hydroxybutyric acid, and injected into a GC/MS for quantification using a standard curve. Cells contain intracellular granules of PHB, which can be extracted using chloroform and other methods. Therefore, extractions using a chloroform-methanol method was used to isolate pure PHB from whole cells. Mass before extraction was measured, and the mass after extraction was measured to determine the total PHB content of the cells and to reach the 60% of cell dry weight (CDW) value. PHB was extracted by taking cell pellets and lyophilizing overnight. Next, boiling acetone was added to the cell pellet and shaken gently until the acetone was evaporated. Next, 50 volumes of chloroform was added to the cell pellet and incubated at 30 C for at least one hour, after which the cell debris was pelleted by centrifugation at 1000 g for 5 minutes. The supernatant was moved to a new vial, then a 7:3 (v/v) methanol: water solution was used to precipitate the PHB and dried to determine the final mass of the extracted PHB. The material was also analyzed using gel permeation chromatography following someone skilled in the art. The PHB material was also characterized by derivatization which can be followed as someone skilled in the art. PHB was converted to 3-methyl hydroxybutyrate by adding 2 ml of a methanolysis solution containing 97% wt. methanol and 3% wt. sulfuric acid and 2 ml of chloroform to cell pellet of H. elongata and heating at 100° C. for 4 h. Benzoic acid was used as an internal standard for derivatization. After derivatization, the chloroform phase was analyzed using gas chromatography coupled with mass spectrometry (GC-MS).

### Example 9: Dye Stripping Analysis

For a typical hydrothermal treatment, 2.5 g of the chosen material (cotton, polyester, or cotton+polyester) was added to a 50 mL aqueous NaOH solution (0.5M) in a 1 L stainless steel autoclave (Parr Instrument Company, 4570 series) equipped with a mechanical stirrer and water cooling coils. The autoclave was heated to the desired reaction temperature (standard conditions are at 180° C.) using an inductive heater (EASYHeat 024 FE CE) and held at temperature for 30 minutes.

These hydrothermal treatments were generally observed to solubilize polyester material, while simultaneously stripping dyes from solubilized material and stripping dyes from residual material as well, like cotton. The above standard cotton dye stripping conditions were applied for red, blue, and pink textiles, each yielding a colored aqueous solution and off-white residual cotton fabric.

To evaluate the extent of dye stripping from cotton, the sample color, including the visible spectrum from 360 nm to 70 nm, was measured on a Datacolor 850 spectrophotometer. The instrument used was a dual beam spectrophotometer, operating in a d/8° geometry (diffuse illumination, measure at 8° from normal to the sample surface), with a pulsed xenon lamp as the light source and a 152 mm integrating sphere. Measurements were made in both specular reflectance included and specular reflectance excluded modes. No UV filter was used on the light source, so samples containing fluorescent brightener dyes exhibited reflectance higher than 100% at some wavelengths. The 9 mm (SAV) aperture was used on the sample port. Fabric samples were folded to make 4 or 8 layers (2 or 4 folds) to create an effectively opaque material. The folded fabric samples were placed against the sample port and measured with the calibration white tile behind them for consistency. The reflectance was recorded at 10 nm intervals from 360 nm to 700 nm. Color coordinates were calculated by the Datacolor Tools software for a D65 illuminant (standard daylight with a 6500 K color temperature) and a 10° observer as defined by the International Commission on Illumination (CIE). Data was exported to a text file for further analysis.

To determine the “closeness” to the expected final color for a hydrothermally treated white cotton fabric, the ΔE metric was used, which expresses the distance between any two colors in CIELAB color space. These values can be expressed by the following equation:

$$Δ {E}_{12} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

where L*, a*, and b* correspond to coordinates of colors 1 and 2 in CIELAB color space. The percent change in color of colored cotton fabrics post hydrothermal treatment was calculated using the following equation:

$$\% change = \frac{Δ {E}_{{C}_{t} {W}_{t}}}{Δ {E}_{{C}_{u} {W}_{t}}}$$

where ΔE_{CtWt} corresponds to the ΔE value for the hydrothermally treated colored t-shirt and a hydrothermally treated white t-shirt, and ΔE_{CtWt} corresponds to the ΔE value for the colored t-shirt prior to treatment and a hydrothermally treated white t-shirt. The ΔE and % change values for each hydrothermally treated cotton t-shirt are shown in Table 3. The red and pink t-shirts demonstrated the largest percent change in their ΔE values, being 90.3% and 92.0%, respectively. While being lower than the values seen for the lighter colored t-shirts, the blue t-shirts also saw significant changes in their ΔE values at 64.6% and 59.4%, respectively. While these % change in ΔE values may be used as a quantitative measure of the change in color of colored cotton t-shirts after hydrothermal treatment for dye stripping, it may not by itself serve as a quantitative measure for the amount of dye removed. The change in color perceived does not correspond linearly to the amount of dye removed as the t-shirts are saturated with dye and require significant amounts of dye removal before perceptible differences in color may be observed. Therefore, the % removal of dye from hydrothermal treatment is likely much higher than the calculated % change in ΔE values, indicating that almost all of the dye has like been removed from the cotton fabrics.

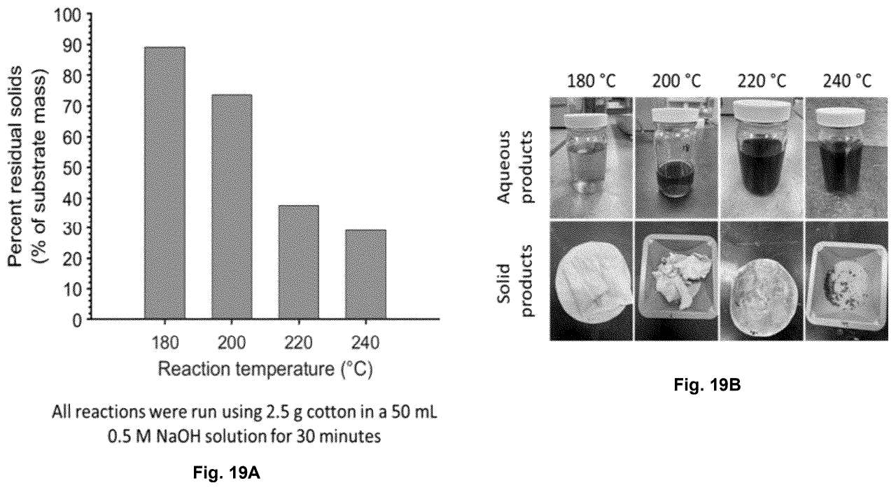

The corresponding visible light spectra obtained for both untreated and treated colored fabrics are shown in FIG. 19. Post hydrothermal treatment, distinctive peaks corresponding to the color of each fabric disappear or are significantly diminished, corresponding to dye removal. For the untreated white fabric, a peak at ˜450 nm with a peak reflectance value at 140% indicates the presence of fluorescent optical brighteners that are common in white clothing. This peak is significantly diminished post hydrothermal treatment, indicating at least partial breakdown of the fluorescent compound.

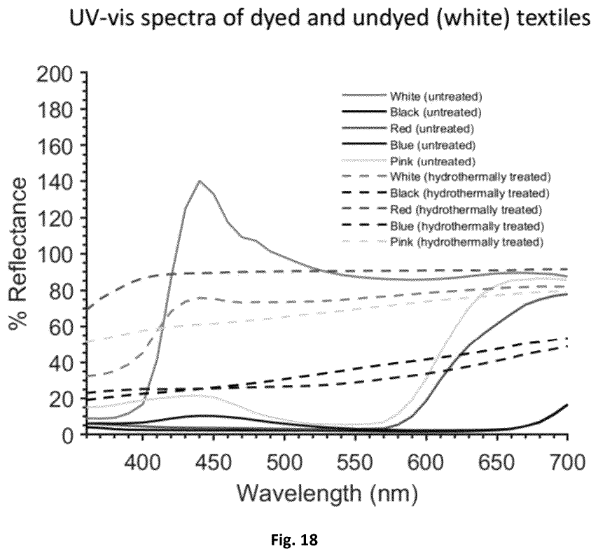

This color analysis was conducted on all hydrothermally treated color fabrics and compared to a hydrothermally treated white cotton fabrics (Table 3). In all cases, this analysis indicated that a significant amount of the dye was stripped from the cotton fabrics during hydrothermal processing. This is corroborated by UV-vis spectra of the fabrics, which demonstrate the disappearance or significant diminishment of all peaks present that would contribute to the perceived color of the fabric (FIG. 18).

TABLE 2
Strain compatibility with common textile
dyes at various concentrations.

| | 0.1 g/L | 0.5 g/L | 1 g/L | 5 g/L |
|---|---|---|---|---|
| Compound | Growth? | Growth? | Growth? | Growth? |
| No Dye | ++ | ++ | ++ | ++ |
| Indigo | ++ | ++ | ++ | + |
| Remazol | ++ | ++ | + | − |
| brilliant blue | | | | |
| Orange II | − | − | − | − |
| Trypan blue | ++ | ++ | ++ | + |
| Indigo | ++ | ++ | ++ | ++ |
| carmine | | | | |

TABLE 3
Color analysis of dyed cotton textile compared to
undyed (white) textile showing drastic change in
color intensity after hydrothermal stripping

| Sample | ΔE(Cᵤ − Wₕ) | ΔE(Cₕ − Wₕ) | % Change ΔE |
|---|---|---|---|
| White | 20.39 | — | — |
| Black | 73.64 | 26.08 | 64.6% |
| Red | 74.44 | 7.20 | 90.3% |
| Blue | 70.67 | 28.69 | 59.4% |
| Pink | 68.79 | 5.49 | 92.0% |

### Example 10: ATR-IR and Gravimetric Analysis of Hydrothermal Treatment Effects on Cotton

To understand the effect of increasing reaction temperature on the fate of cotton present during hydrothermal treatment, we investigated the treatment cotton textile samples with 0.5M NaOH in water at 180, 200, 220, and 240° C., and compared to untreated cotton. Residual solids were recovered from the reactor, dried, and weighed to gravimetrically evaluate mass loss. These samples were then evaluated with IR analysis using an Agilent Cary 670 Fourier transform infrared spectrometer outfitted with a Pike GladiATR attenuated total reflectance (ATR) accessory. ATR-IR spectra were obtained against a diamond crystal and analyzed at 4 cm⁻¹ wavenumber resolution for the range of 4000 to 400 cm⁻¹ wavenumbers.

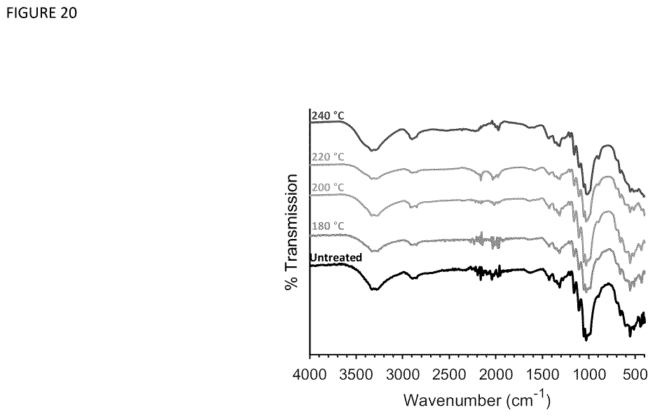

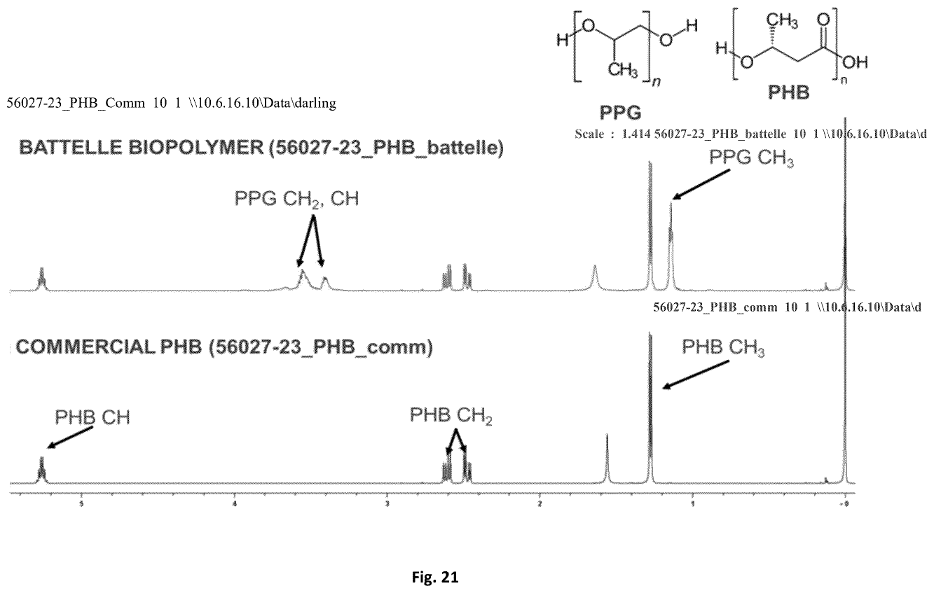

Cotton treatment at 180° C. yielded an off-white cotton fabric with only 10.8+1.1% mass loss from the original substrate (FIG. 19). Increasing the reaction temperature increased the extent of charring of the cotton substrate significantly as well as the overall solids mass loss, with treatment at 240° C. yielding brown flakes with at 71% mass loss. ATR-FTIR spectra for untreated and hydrothermally treated white cotton fabrics were nearly identical, notably with a reduction in the intensity of the broad peak at ˜1600 cm⁻¹ (FIG. 20). This may be attributed to the breakdown of the fabric whiteners that are commonly found in white textiles. IR spectroscopy of the residual solids demonstrated some increasing signs of oxidation at higher reaction temperatures with the emergence of peaks in the 1900-2200 cm⁻¹ region. From these results, it was evident that hydrothermal treatment at 180° C. provided a balance between achieving high yields of polyester depolymerization and minimization of cotton charring. It is for this reason that processing at 180-200° C. in 0.5M NaOH for 30 minutes was selected as preferred processing conditions for the treatment of cotton/polyester blends.

### Example 11: Characterization of the Large Scale PHB

### NMR Characterization (56027-23, 56027-25)

NMR characterization of Battelle's poly-3-hydroxybutyrate (PHB) sample indicated the presence of both PHB and polypropylene glycol in a 59:41 wt. % ratio. The PPG is a residual foaming agent from the fermentation process that was not completely removed. In order to compare different regions of the PHB sample to check for possible heterogeneity, 3 different samples from the PHB material were characterized by NMR. Comparison of the ¹H NMR spectrum from these different samples indicated a near uniform ratio of PHB and PPG across the entire sample.

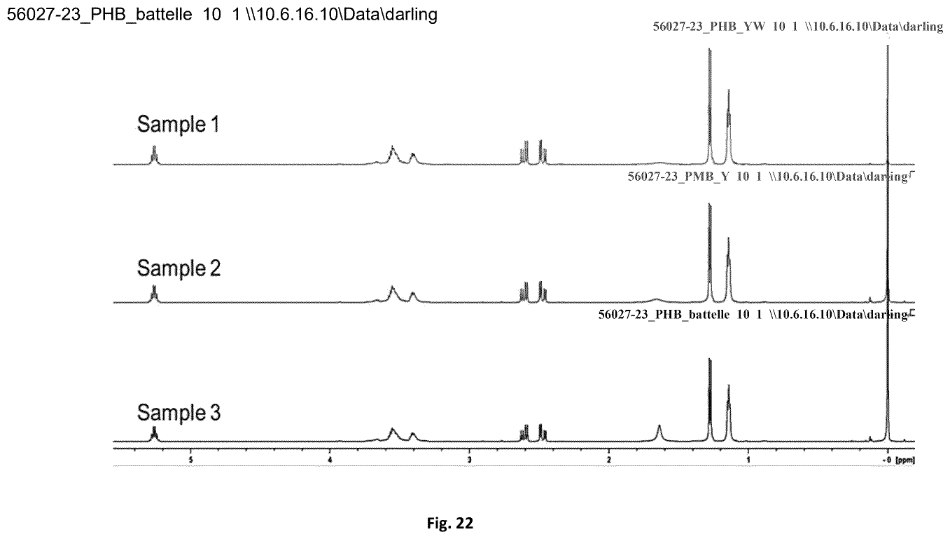

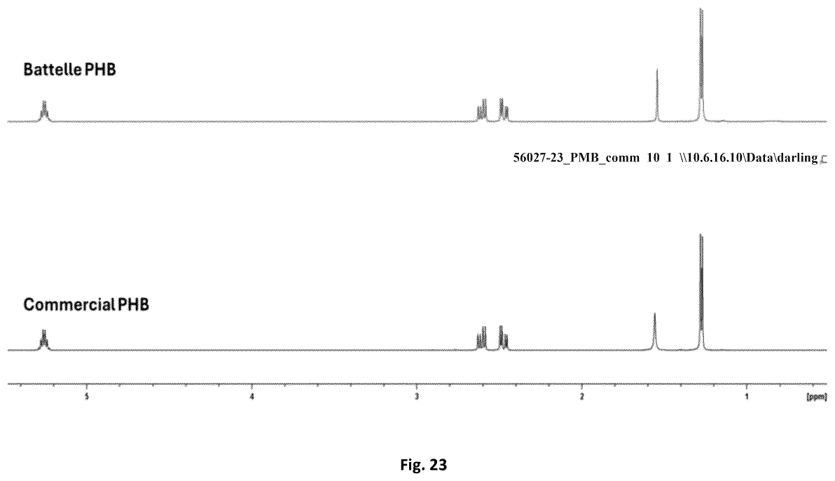

PPG was removed from PHB via selective precipitation. After 2 successive selective precipitations, the wt. % ratio of PHB to PPG changed from 59:41 to 98:2. The NMR for the purified Battelle PHB and Commercial PHB are shown in (FIG. 23)

TABLE 4
Ratio of PHB and PPG across different areas of the large
scale Battelle PHB sample as indicated by 1H NMR.

| Sample | PHB:PPG wt. % ratio |
|---|---|
| 1 | 58:42 |
| 2 | 59:41 |
| 3 | 59:41 |

### Example 12: GPC Characterization

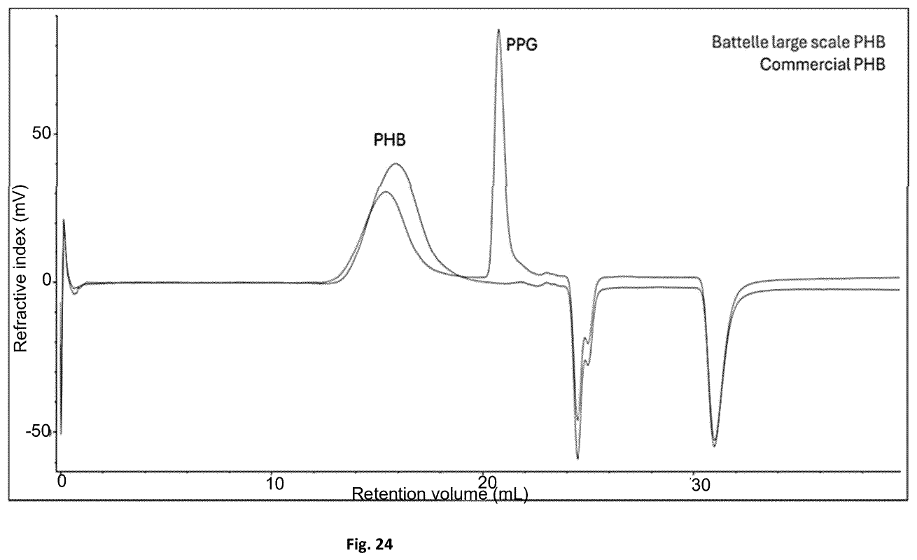

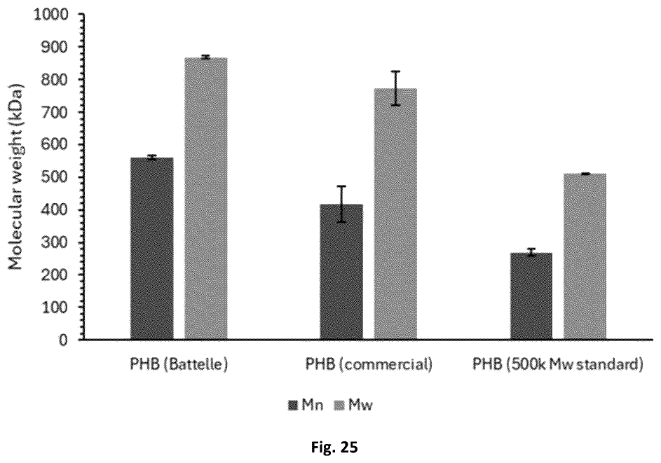

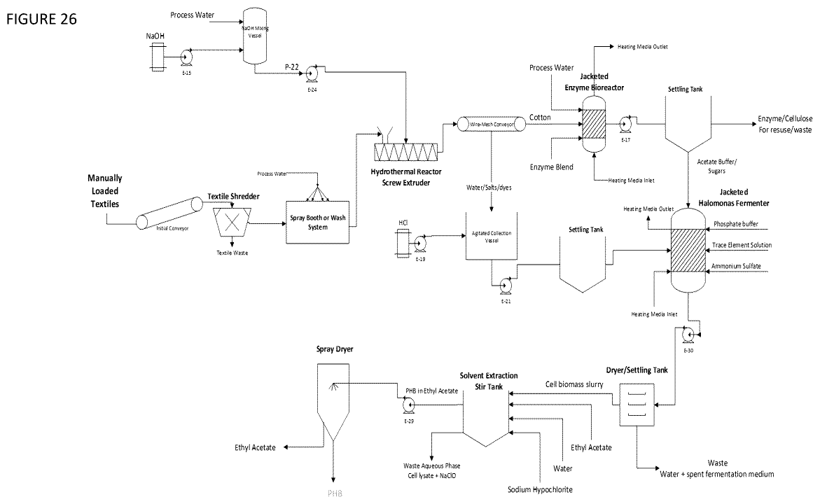

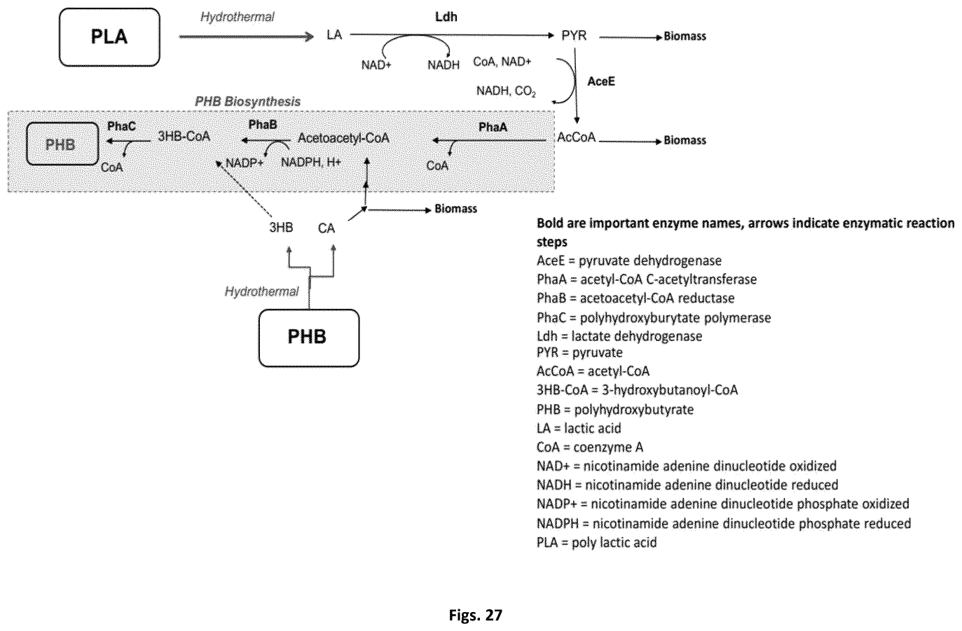

GPC characterization of Battelle's large scale PHB sample, a commercial PHB sample from Sigma Aldrich, and a 500 KDa PHB control from Sigma Aldrich was conducted. GPC characterization demonstrated the presence of PHB and PPG as distinct peaks at approximately 16 and 21 minutes for the large scale Battelle PHB sample, while only the PHB peak appeared for the commercial PHB sample (FIG. 24). The relative Mn and Mw values for these samples, as well as a 500 KDa M_{w} PHB sample are shown in (FIG. 25) Overall, Battelle's PHB sample demonstrated Mₙ and M_{w} values of 560 and 870 KDa.

### Example 13: Mechanical Properties Characterization

Mechanical characterization of films prepared via tensile testing with both commercial PHB and Battelle PHB post-PPG extraction (Table 5). Tensile test specimens were prepared via solvent casting of PHB and cutting dog bones from the resulting films. Tensile tests were performed at room temperature at a stretching speed of 5 mm/min. The tensile strength and elongation at break were similar to that of Battelle's PHB sample. The Young's modulus of the Battelle PHB sample was lower than that of commercial PHB. This may be attributed to the 2 wt. % PPG found within the PHB sample, which acts as a plasticizer.

TABLE 5
Tensile test results for both commercial and Battelle PHB

| | Young's | Tensile | Elongation |
|---|---|---|---|
| Sample | modulus (MPa) | strength (MPa) | at break (%) |
| Commercial PHB | 1452 | 15.2 | 2.16% |
| Battelle PHB | 784 ± 319 | 11.6 ± 4.8 | 1.76 ± 0.29% |

### Example 14: Hydrothermal Depolymerization of PLA and PHB

The following is a set of reaction conditions that can be generalizable for the co-hydrothermal treatment of PLA and PHB:

As illustrated in FIG. 28, a 1:1 mixture of PLA and PHB resin (usually 2 g each) was added to 50 mL H₂O in a beaker. A basic (NaOH) additive (1 g) was added to the reaction solution and heated to a reaction temperature of 180° C.) and held for the duration of the reaction (30 minutes). Upon completion of the reaction, the reaction contents were removed. The collected residual solids were collected using filter paper and a separatory funnel and dried. The mass of the residual solids was measured and recorded. The filtrate, indicated depolymerization products and the assumed percent depolymerization was quantified.

### Example 15: Detection of Hydrothermal Breakdown Products

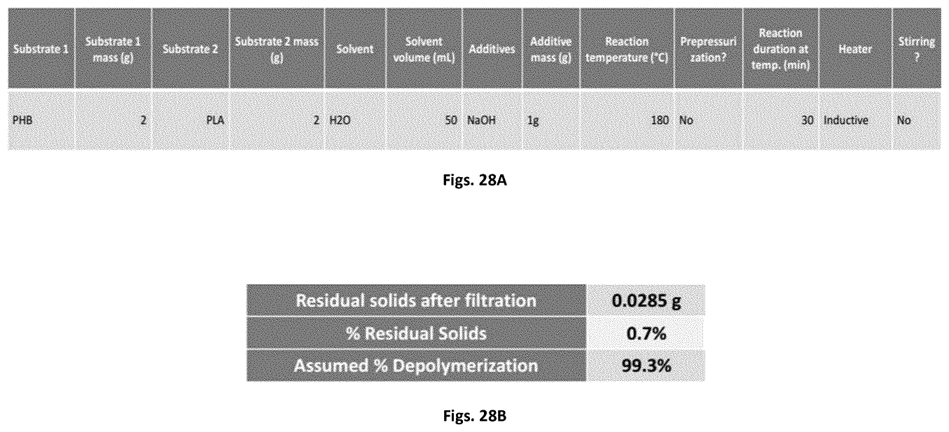

As illustrated in FIG. 29, LC/MS was performed using a SCIEX TripleTOF 6600 in negative ion mode. Samples were diluted 1000-fold for measurement and concentrations were calculated by converting observed peak areas to concentrations using a standard curve for each compound. Each standard curve contained at least six points and calibration curves were calculated using regression equations. R² values for calibration curves were >0.99 for each compound.

### Example 16: Growth of Halomonas elongata on Lactate and Crotonate as the Carbon Source

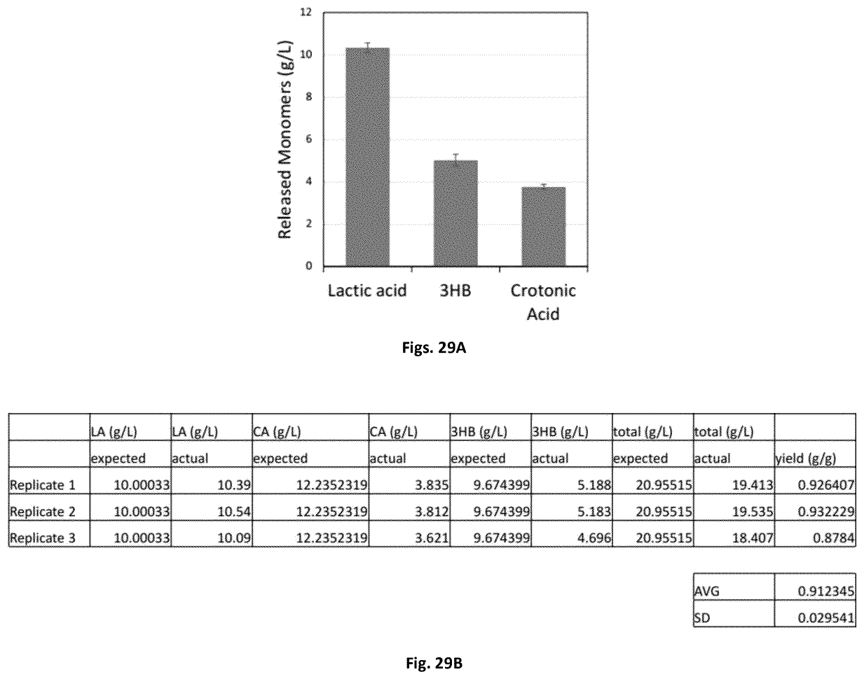

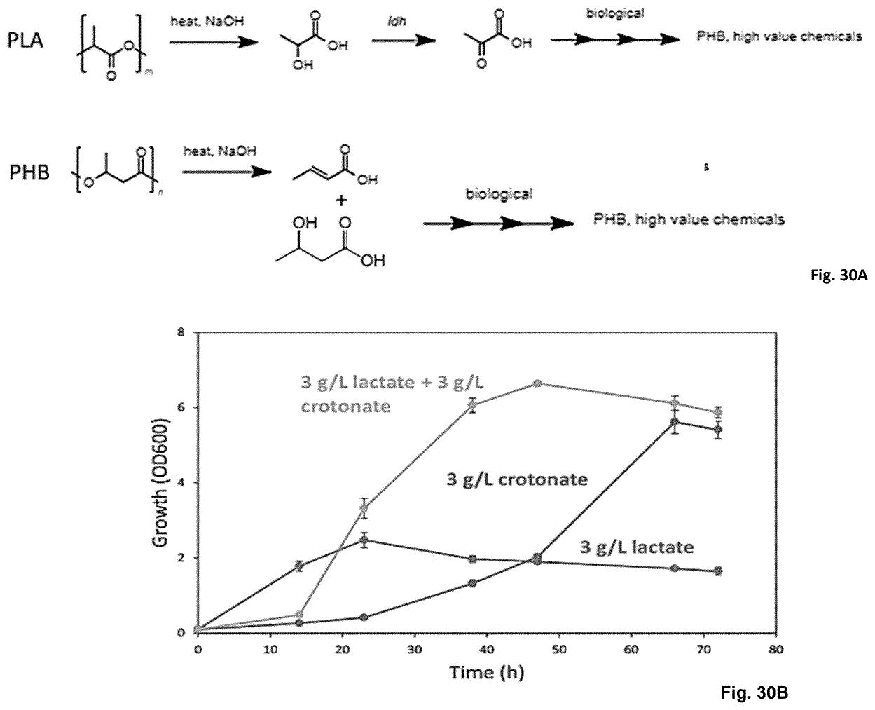

As demonstrated in FIG. 30B, the growth of Halomonas elongata on 3 g/L lactic acid or 3 g/L crotonic acid or 3 g/L lactic acid+3 g/L crotonic acid was investigated. Media was prepared with MM63 minimal salts medium directly and used as a growth substrate for Halomonas elongata strain. MM63 minimal salts medium contains 13.61 g/L KH·PO₄, 4.21 g/L KOH, 1.98 (NH₄)₂SO₄, 0.0011 g/L FeSO₄—H2O 0.25 g/L MgSO₄-7H2O, 60 g/L NaCl. A final optical density of 6.0 was achieved in media containing a combination of crotonate and lactate or crotonate alone. Assuming linearity in biomass to substrate loading, and assuming the major carbon source was crotonate and/or lactate, the increased optical density over time would indicate a high conversion of the substrates in the Halomonas strain.

### Cell Density:

Cell density was measured using a spectrophotometric assay detecting absorbance of each well/cuvette at 600 nm. The measurements were performed using a Thermo Scientific Genesys 150 spectrophotometer. A non-inoculated control was used to subtract background absorbance.

Minimal salts medium: 13.61 g/L KH·PO₄, 4.21 g/L KOH, 1.98 (NH₄)₂SO₄, 0.0011 g/L FeSO₄—H2O 0.25 g/L MgSO₄-7H2O, 60 g/L NaCl.

### Example 17: Halomonas Utilization of Hydrothermal Treatment Reaction Products of PLA and PHB

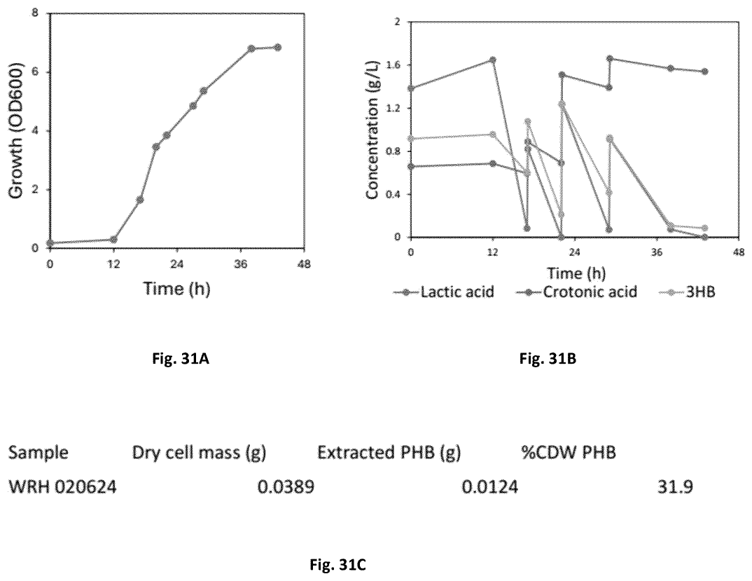

We also explored the growth of Halomonas elongata on reaction products from the hydrothermal treatment of PLA and PHB. This was prepared with MM63 minimal salts medium directly and used as a growth substrate for the WT Halomonas elongata strain. The reaction products were added directly to the medium after pH adjustment to basic pH, and the medium was adjusted to a final pH of 8.0 (FIG. 31).

An increase in cell density over the 48 h growth, as measured by the optical density revealed supportive growth conditions afforded by reaction products.

### Example 18: Characterization of PHB

As shown in FIG. 31, the polyhydroxybutyrate polymer was derivatized into methyl-3-hydroxybutyric acid, and injected into a GC/MS for quantification using a standard curve. Cells contain intracellular granules of PHB, which can be extracted using chloroform and other methods. Therefore, extractions using a chloroform-methanol method was used to isolate pure PHB from whole cells. Mass before extraction was measured, and the mass after extraction was measured to determine the total PHB content of the cells and to reach the 32% of cell dry weight (CDW) value. PHB was extracted by taking cell pellets and lyophilizing overnight. Next, boiling acetone was added to the cell pellet and shaken gently until the acetone was evaporated. Next, 50 volumes of chloroform was added to the cell pellet and incubated at 30° C. for at least one hour, after which the cell debris was pelleted by centrifugation at 1000 g for 5 min. The supernatant was moved to a new vial, then a 7:3 (v/v) methanol: water solution was used to precipitate the PHB and dried to determine the final mass of the extracted PHB. The material was also analyzed using gel permeation chromatography. The PHB material was also characterized by derivatization. PHB was converted to 3-methyl hydroxybutyrate by adding 2 ml of a methanolysis solution containing 97% wt. methanol and 3% wt. sulfuric acid and 2 ml of chloroform to cell pellet of H. elongata and heating at 100° C. for 4 h. Benzoic acid was used as an internal standard for derivatization. After derivatization, the chloroform phase was analyzed using gas chromatography coupled with mass spectrometry (GC-MS).

### Example 19: Hydrothermal Depolymerization of PLA and PET Contaminated with Food

Hydrothermal deconstruction experiments on mixed packaging material consisting of PLA and PET were performed with and without peanut butter. A summary of the experiment is shown below in Table 6. Conditions for deconstruction were similar to previous studies of 180° C. for 30 min, with 0.5M NaOH. High breakdown was observed of >95% for all experiments as determined by gravimetric analysis of residual solids, consistent with previous studies. Filtered aqueous hydrolysate, denoted as “Aqueous fraction” in Table 6 was subsequently prepared for microbial upcycling.

TABLE 6
Summary of hydrothermal deconstruction experiments of mixed food and plastic input.

| | Substrate | Solvent | | Temperature | Time | Residual | Aqueous |
|---|---|---|---|---|---|---|---|
| Substrate | mass (g) | (g) | Additive | (° C.) | (min) | solids (%) | fraction (g) |
| Plastic | 1 g PET, 1 g | 50 g | 1 gram | 180° C. | 30 min | −0.3% | 47.18 g |
| | PLA | water | NaOH | | | | |
| Food | 0.5 g peanut | 50 g | 1 gram | 180° C. | 30 min | −0.3% | 43.05 g |
| | butter | water | NaOH | | | | |
| Plastic + | 1 g PET, 1 g | 50 g | 1 gram | 180° C. | 30 min | 4.1% | 48.73 g |
| Food | PLA, 0.5 g | water | NaOH | | | | |
| | peanut butter | | | | | | |

### Example 20: Halomonas Utilization of Mixed Food and Plastic Hydrolysate

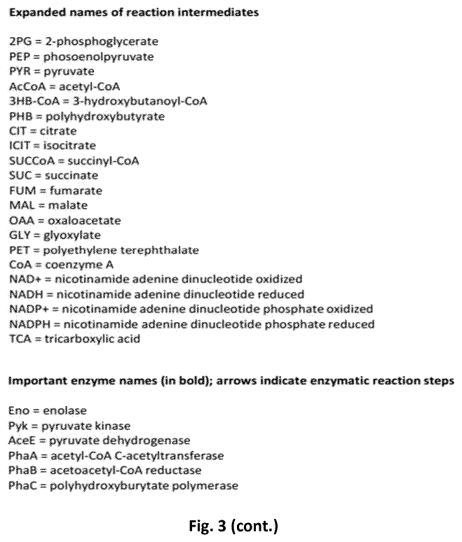

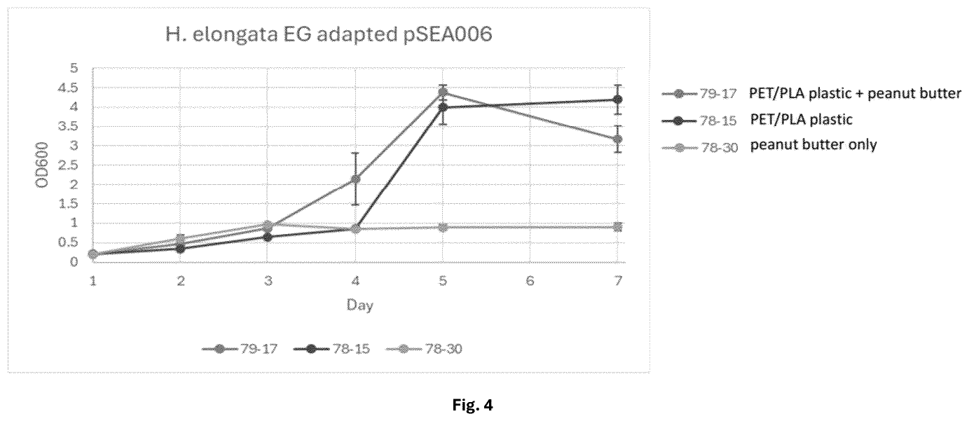

To evaluate microbial upcycling of mixed food and plastic hydrolysate, the engineered strain (Halomonas elongata EG-adapt pSEA006) was inoculated in baffled flasks of 25 mL culture to an OD600 of 0.2 in MM63 media. 6.25 mL of deconstruction products was added to each tube as a carbon source. A control without inoculation was also prepared. The starting OD600 of each flask was recorded, and flasks were incubated at 37° C. and 225 rpm, taking ODs once daily. Once stationary phase was reached, each was spiked with an additional 1 ml of appropriate deconstruction hydrolysate, and OD600 was measured periodically. As shown in FIG. 4, a robust growth after a 3 day lag phase on plastic-only and plastic+peanut butter samples was observed, with similar growth rates observed between these conditions, suggesting food contamination does not inhibit the upcycling process. Slight growth was observed on peanut butter-only condition, likely indicating that only a small amount of upgradeable hydrocarbons were liberated during hydrothermal treatment of peanut butter.

### Example 21: PHB Extraction from Utilization of Mixed Food and Plastic Hydrolysate

PHB extraction was performed on these samples, using solvent extraction approach as described in previous examples. PHB isolated from these experiments is shown in Table 7. We observe the % PHB content of cells ranging from 16-29%, demonstrating that deconstruction products were converted to PHB. Further optimization and scale-up of growth and media conditions would enable higher PHB production from deconstruction products.

TABLE 7
Summary of PHB extraction results for selected samples

| Sample Input | Sample Code | % CDW PHB content |
|---|---|---|
| PET/PLA/PB | 79-17 Flask 1 | 16.9 |
| PET/PLA/PB | 79-17 Flask 2 | 15.5 |
| PB | 78-30 Flask 4 | 28.6 |
| PB | 78-30 Flask 5 | 25.0 |

1. A method for deconstructing polyester cotton blended textile comprising:
alkaline hydrothermal treatment, conducted at a pH of more than pH 7 and at a temperature of at least 180° C., of polyester cotton blended textile to generate TPA and EG from the polyester, depolymerizing cotton, forming breakdown products; and
contacting the breakdown products with bacteria of the genus Halomonas in a fermentation bioreactor.

2. The method of claim 1, wherein said hydrothermal treatment is conducted at a pH of pH 7.5 to 11 and at a temperature of 180° C. to 210° C.

3. The method of claim 1 wherein the polyester is polyethylene terephthalate.

4. The method of claim 1 wherein the polyester comprises at least one polyester containing fiber.

5. (canceled)

6. An engineered microbial cell that utilizes TPA to produce PHB.

7. The engineered microbial cell of claim 6, wherein the engineered microbial cell expresses non-native TphA1II.

8. The engineered microbial cell of claim 6, wherein the engineered microbial cell expresses non-native TphA2II or expresses non-native TphA3II.

9. The engineered microbial cell of claim 6, wherein the engineered microbial cell expresses non-native TphBII.

10. The engineered microbial cell of claim 6, wherein the engineered microbial cell expresses non-native TphCII.

11. The engineered microbial cell of claim 6, wherein the engineered microbial cell expresses non-native TphRII.

12. The engineered microbial cell of claim 6 that comprises increased activity of at least one or more upstream pathway enzyme(s) leading to improved ethylene glycol utilization, said increased utilization being increased relative to a control cell.

13. The engineered microbial cell of claim 6, wherein the engineered microbial cell is a bacterial cell.

14. The engineered microbial cell of claim 13, wherein the engineered microbial cell is of the genus Halomonas.

15. The engineered microbial cell of claim 13, wherein the engineered microbial cell is of the species elongata.

16. The engineered microbial cell of claim 13, wherein the engineered microbial cell converts glucose and produces at least 50% or 50% to 65% or 50% to 60% of cellular dry weight as PHB.

17-35. (canceled)

36. A method for deconstructing a mixture comprising polyester, biodegradable polymer and food comprising:
alkaline hydrothermal treatment, conducted at a pH of more than pH 7 and at a temperature of at least 180° C., forming breakdown products; and
contacting the breakdown products with engineered bacteria of the genus Halomonas in a fermentation bioreactor.

37. The method of claim 36, wherein said hydrothermal treatment is conducted at a pH of pH 7.5 to 11 and at a temperature of 180° C. to 210° C.

38. The method of claim 36, further comprising the cell of claim 6.

39. The microbial cell of claim 38 wherein hydrothermal breakdown products of polyester, biodegradable polymer and food produces at least 15% or 15 to 30% or 20 to 50% of cellular dry weight as PHB.

40. The method of claim 1 wherein the alkaline hydrothermal treatment comprises removing dyes from blended textile.

41-43. (canceled)
